SEQUENCE LISTING (4 sequences)

SEQ ID NO: 1 - DNA, 6 bp, Homo sapiens
ttaggg

SEQ ID NO: 2 - DNA, 11 bp, Homo sapiens
gttagggttag

SEQ ID NO: 3 - DNA, 11 bp, Homo sapiens
ctaaccctaac

SEQ ID NO: 4 - DNA, 11 bp, Homo sapiens
gatcgatcgat

(19) United States
(12) Patent Application Publication
(10) Pub. No.: US 2006/0269924 A1
(43) Pub. Date: Nov. 30, 2006

(54) Modulation of telomere-initiated cell signaling

(75) Inventors: Barbara A Gilchrest, Boston, MA (US); Mark S. Eller, Boston, MA (US)

Correspondence Address: HOWREY LLP, C/O IP DOCKETING DEPARTMENT, 2941 FAIRVIEW PARK DR, SUITE 200, FALLS CHURCH, VA, 22042-2924, US

(73) Assignee: TRUSTEES OF BOSTON UNIVERSITY, Boston, MA (US)

(21) Appl. No.: 10/553,001

(22) PCT Filed: Jan. 14, 2004
(86) PCT No.: PCT/US04/00819

§ 371 Date: Jul. 24, 2006

(30) Foreign Application Priority Data
Apr. 11, 2003 (WO) PCT/US03/11393

(51) Int. Cl. A61K 48/00; C12Q 1/68; G01N 33/574; A61K 38/54

(52) U.S. Cl. 435/6; 435/7.23; 514/44; 514/12

(57) ABSTRACT

The use of modulators of Mre 11, tankyrase, the DNA damage pathway and MRN complex formation of the protection of mammals from failure of growth arrest, apoptosis or proliferative senescence.

### CROSS-REFERENCES TO RELATED APPLICATIONS

This application is a continuation-in-part of International Application No. PCT/US03/11393, filed Apr. 11, 2003, the contents of which are hereby incorporated in their entirety.

### BACKGROUND OF THE INVENTION

1. Field of the Invention

The present invention relates to the regulation of signaling pathways. More specifically, the present invention relates to the regulation of telomere-initiated senescence, apoptosis, tanning and other DNA damage responses.

2. Description of Related Art

The frequency of cancer in humans has increased in the developed world as the population has aged. For some types of cancers and stages of disease at diagnosis, morbidity and mortality rates have not improved significantly in recent years in spite of extensive research. During the progression of cancer, tumor cells become more and more independent of negative regulatory controls, including resistance to senescence and apoptosis, the important aspects of how the interaction of normal cells with their tissue-specific environment is regulated.

Cellular senescence has been suggested to be an important defense against cancer. Extensive evidence implicates progressive telomere shortening or telomere dysfunction caused by an inability to replicate the 3′ ends of chromosomes in senescence. In germline cells and most cancer cells, immortality is associated with maintenance of telomere length by telomerase, an enzyme complex that adds TTAGGG repeats to the 3′ terminus of the chromosome ends. Telomeres, tandem repeats of TTAGGG, end in a loop structure with a 3′ single-stranded overhang of approximately 150-300 bases tucked within the proximal telomere duplex DNA and stabilized by telomeric repeat binding factors (TRFs), particularly TRF2. Ectopic expression of a dominant-negative form of TRF2 (TRF2^{DN}) disrupts telomere loop structure, exposes the 3′ overhang and causes DNA damage responses, followed by senescence in primary fibroblasts and fibrosarcoma cells.

Senescence can also be precipitated acutely by extensive DNA damage or the overexpression of certain oncogenes. Ectopic expression of the telomerase reverse transcriptase catalytic subunit (TERT), which enzymatically maintains or builds telomere length, can bypass senescence with subsequent immortalization of some human cell types, strongly suggesting a telomere-dependent mechanism of replicative senescence. Moreover, malignant cells commonly express TERT and/or contain mutations that allow the cell to bypass the senescent response and to proliferate indefinitely despite often having shorter telomeres than normal senescent cells. However, some tumor cells undergo senescence in response to various anticancer agents, indicating that acquisition of immortality does not necessarily imply a loss of this basic cellular response to DNA damage.

Senescence in human cells is largely dependent on the p53 and pRb pathways. The tumor suppressor p53 plays a key role in cellular stress response mechanisms by converting a variety of different stimuli, for example, DNA damage, deregulation of transcription or replication, oncogene transformation, and deregulation of microtubules caused by some chemotherapeutic drugs, into cell growth arrest or apoptosis. When activated, p53 causes cell growth arrest or a programmed, suicidal cell death, which in turn acts as an important control mechanism for genomic stability. In particular, p53 controls genomic stability by eliminating genetically damaged cells from the cell population, and one of its major functions is to prevent tumor formation.

An intact tumor suppressor pRb pathway is needed to prevent tumorigenesis. In pRb^{-/-} tumor cells that do not contain wild-type p53, introduction of pRb induces senescence. Although cervical cancer cells frequently retain wild-type p53 and pRb genes, the HPV E6 and E7 proteins interfere with the p53 and pRb pathways, respectively. Ectopic expression of viral E2 protein represses HPV E6 and E7 gene transcription and induces a rapid and prominent senescent response in cervical carcinoma cell lines, again affirming the important roles of p53 and pRb in cancer cell senescence.

Suppressing only the p53 or the pRb pathway is not sufficient for fibroblasts to bypass replicative senescence. Indeed, human fibroblasts either transfected with SV 40 T antigen or transduced with combinations of adenovirus E1A+E1B or HPV E6+E7, suppressing both the p53 and pRb pathways, have an extended life span and escape replicative senescence.

Double strand breaks in DNA are extremely cytotoxic to mammalian cells. The highly conserved MRN complex is involved in the repair of double strand breaks in eukaryotes. The MRN complex adheres to sites of double strand breaks immediately following their formation. The MRN complex also migrates to telomeres during the S-phase of the cell cycle associates with telomeric repeat binding factors (TRF).

The MRN complex consists of Mre11, Rad50 and NBS (p95). Mre11, as part of the Mre11/p95/Rad50 complex, associates with the telomere 3′ overhang DNA during S phase of the cell cycle. Mre11 is an exonuclease with preference for the 3′ end of a DNA strand. The activity of Mre11 is believed to be dependent on interaction with Rad50, which is an ATPase. Nbs1 is believed to be involved in the nuclear localization of the MRN complex, as-well as its assembly at the site of a double strand break.

Cancers are typically treated with highly toxic therapies, such as chemotherapy and radiation therapy, that comparably damage all proliferative cells whether normal or malignant. Side effects of such treatments include severe damage to the lymphoid system, hematopoietic system and intestinal epithelia, as well as hair loss. Other side effects include hair loss. There continues to be a need for safer and more effective cancer therapies, especially for alternative therapies that would avoid some or all of these side effects by preferentially targeting malignant cells relative to normal but proliferative cells.

### SUMMARY OF THE INVENTION

The present invention relates to an in vitro method of screening for a modulator of Mre11 comprising contacting candidate modulators with Mre11 in vitro in the presence of a nucleic acid substrate for Mre11, and measuring the hydrolysis of the substrate. A modulator may be identified by altering hydrolysis of the substrate nucleic acid compared to a control. The nucleic acid substrate may be an oligonucleotide with at least 50% nucleotide sequence identity with (TTAGGG)ₙ, wherein n=1 to 20. The hydrolysis of the substrate nucleic acid may be measured by UV absorbance, gel analysis of labeled oligos, or recovery of non-precipitatable nucleotide bases.

The present invention also relates to an in vitro method of screening for an agent that specifically binds to Mre11, comprising contacting candidate agents with Mre11, and determining whether a candidate agent specifically binds to Mre11. Mre11 may be attached to a solid support.

The present invention also relates to a cell-based method of screening for a modulator of Mre11, comprising contacting candidate modulators with a cell that expresses Mre11 under conditions in which the modulator is taken up by the cell, and measuring a property of the cells including, but not limited to, cellular proliferation, cellular viability, cellular morphology, SA-β-Gal activity and phosphorylation of p53 or p95. A modulator may be identified by altering the property compared to a control. The candidate modulator may be an agent that specifically binds to Mre11 as identified above. Mre11 may be expressed as a fragment, homolog, analog or variant of Mre11, which may have exonuclease-activity.

The present invention also relates to an in vitro method of screening for a modulator of tankyrase comprising contacting candidate modulators with tankyrase in vitro in the presence of a substrate for tankyrase, and measuring the ribosylation of the substrate. A modulator may be identified by altering ribosylation of the substrate compared to a control. The substrate may be a peptide or polypeptide, which may be TRF. The ribosylation of the substrate may be measured by UV absorbance or labeling-of the substrate.

The present invention also relates to an in vitro method of screening for an agent!that specifically binds to tankyrase, comprising contacting candidate agents with tankyrase, and determining whether a candidate agent specifically binds to tankyrase. Tankyrase may be attached to a solid support.

The present invention also relates to a cell-based method of screening for a modulator of tankyrase, comprising contacting candidate modulators with a cell that expresses tankyrase under conditions in which the modulator is taken up by the cell, and measuring a property of the cells including, but not limited to, cellular proliferation, cellular viability, cellular morphology, SA-β-Gal activity and phosphorylation of p53 or p95. A modulator may be identified by altering the property compared to a control. The candidate modulator may be an agent that specifically binds to tankyrase as identified above. Tankyrase may be expressed as a fragment, homolog, analog or variant of tankyrase, which may have ribosylase activity.

The present invention also relates to an in vitro method of screening for a modulator of MRN complex formation comprising contacting candidate modulators with Mre11, Rad50 and Nbs1 in vitro, and measuring the formation of the MRN complex. A modulator may be identified by altering formation of the MRN complex compared to a control. Candidate modulators may be contacted with Mre11, Rad50 and Nbs1 in the presence of a nucleic acid substrate or inhibitor of Mre11. The nucleic acid may be an oligonucleotide with at least 50% nycleotide sequence identity with (TTAGGG)ₙ, wherein n=1 to 20. Formation of the MRN complex may be measured by centrifugation, coprecipitation or nondenaturing electrophoresis.

The present invention also relates to a cell-based method of screening for a modulator of the DNA damage pathway, comprising contacting candidate modulators with a cell that expresses Mre11 and tankyrase in the presence of an oligonucleotide under conditions in which the modulator is taken up by the cell, and measuring a property of the cells including, but not limited to, cellular proliferation, cellular viability, cellular morphology, SA-b-Gal activity and phosphorylation of p53 or p95. A modulator may be identified by altering the property compared to a control. The oligonucleotide may have at least 50% nucleotide sequence identity with (TTAGGG)ₙ, wherein n=1 to 20. Mre11 may be expressed as a fragment, homolog, analog or variant of Mre11, which may have exonuclease activity. Tankyrase may be expressed as a fragment, homolog, analog or variant of tankyrase, which may have ribosylase activity.

The cell used in the cell-based screening methods described above may be a cancer cell. The cell used in the cell-based screening methods described may maintain telomeres by telomerase reverse transcriptase or the ALT pathway. The candidate modulators and agents described in the in vitro and cell-based screening methods above may be carbohydrates, monosaccharides, oligosaccharides, polysaccharides, amino acids, peptides, oligopeptides, polypeptides, proteins, nucleosides, nucleotides, oligonucleotides, polynucleotides, lipids, retinoids, steroids, glycopeptides, glycoproteins, proteoglycans, or small organic molecules.

The present invention also relates to the use of compositions comprising an activator of Mre11, tankyrase, the DNA damage pathway or MRN complex formation. The activator may be used to treat cancer, inducing apoptosis, inducing cellular senescence, inhibiting tanning, promoting cellular differentiation or promoting immunosuppression. The activator may be an oligonucleotide activator of Mre11, which may have at least 50% nucleotide sequence identity with (TTAGGG)ₙ, wherein n=1 to 20. From about one to about ten of the first 3′-nucleotide linkages may be hydrolyzable by a 3′ to 5′ nuclease.

The present invention also relates to the use of compositions comprising an inhibitor of Mre11, tankyrase, the DNA damage pathway or MRN complex formation. The inhibitor may be used to inhibit apoptosis, inhibit cellular senescence, promote growth, promote tanning, inhibit cellular differentiation, reduce cancer treatment side effects. The composition may be given in combination with chemotherapy or ionizing radiation. The inhibitor may be an oligonucleotide inhibitor of Mre11, which may have at least 50% nucleotide sequence identity with (TTAGGG)ₙ, wherein n=1 to 20. From about zero to about ten of the first 3′-nucleotide linkages may be hydrolyzable by a 3′ to 5′ nuclease.

The present invention also relates to a composition comprising an oligonucleotide with at least 50% nucleotide sequence identity with (TTAGGG)ₙ and with at least one nonhydrolyzable internucleotide linkage, wherein n=1 to 20. From one to about ten of the first 3′-nucleotide linkages may-be hydrolyzable by a 3′ to 5′ nuclease,-such as Mre11. The oligonucleotide may have at least 50% nucleotide sequence identity with TTAGGG. The oligonucleotide may also have the sequence GTTAGGGTTAG. The nonhydrolyzable linkage may be a phosphorothioate. The oligonucleotide may be a PNA.

### BRIEF DESCRIPTION OF THE DRAWINGS

FIGS. 1A-1H show FACS analysis of propidium iodide stained Jurkat cells (immortalized T lymphocytes), treated with diluent (FIGS. 1A and 1E); 40 μM 11mer-1 pGTTAGGGTTAG (SEQ ID NO: 2) (FIGS. 1B and 1F); 40 μM 11mer-2 pCTAACCCTAAC (SEQ ID NO: 3) (FIGS. 1C and 11G); 40 μM 11mer-3 pGATCGATCGAT (SEQ ID NO: 4) (FIGS. 1D and 1H). Jurkat cells were treated with the stated reagents for 48 hours before analysis (FIGS. 1A-1D) or 72 hours (FIGS. 1E-1H).

FIGS. 2A-2F are profiles showing the results of fluorescence activated cell sorting, for the following additions to the cells: FIG. 2A, diluent; FIG. 2B, 0.4 μM 11mer-1; FIG. 2C, 0.4 μM 11mer-1-S; FIG. 2D, diluent; FIG. 2E, 40 μM 11mer-1; FIG. 2F, 40 μM 11 mer-1-S.

FIGS. 3A-3G are profiles showing the results of fluorescence activated cell sorting, for the following additions to the cells: FIG. 3A, diluent; FIG. 3B, 10 μM 11mer-1; FIG. 3C, 10 μM 11mer-1 and 1 μM 11mer-1-S; FIG. 3D, 10 μM 11mer-1 and 5 μM 11mer-1-S; FIG. 3E, 10 μM 11mer-1 and 10 μM 11mer-1-S; FIG. 3F, 20 μM 11mer-1-S; FIG. 3G, 10μM 11mer-1-S.

FIG. 4 is a bar graph showing the melanin content (in pg/cell) of cells treated with diluent, pTpT or pTspT.

FIG. 5 is a bar graph showing the melanin content (in pg/cell) of cells treated with diluent, 11mer-1 or 11mer-1-S.

FIG. 6 is a bar graph showing the melanin content (in pg/cell) of cells that have been sham-treated (no irradiation, no oligonucleotides), or treated with ultraviolet light (UV), or unirradiated but given pTspT, or irradiated with UV and given pTspT.

FIG. 7 is a diagram of oligonucleotides of nucleotide sequence SEQ ID NO: 2 which were synthesized with phosphorothioate linkages.

FIG. 8 is a bar graph showing the results of testing the effects of phosphorothioate oligonucleotides 1, 2, 3 and 4 depicted in FIG. 7 in causing senescence in cultures of normal neonatal human fibroblasts, indicated by the cells staining positive for β-galactosidase activity. Oligonucleotide “11-1” indicates fibroblast cultures treated with SEQ ID NO: 2 synthesized entirely with phosphodiester linkages. “Dil” indicates fibroblast cultures treated with diluent not containing oligonucleotide.

FIGS. 9-11 demonstrate that downregulating Mre11 protein levels blocks response of T-oligos.

FIG. 12 demonstrates that the p53 and pRb pathways both contribute to T-oligo-induced senescence in human fibroblasts. FIG. 12a: Immunoblot analysis of p53DD and cdk4^{R24C} expression. Cells were collected for protein analysis by western blot using 30 μg total protein and probed for total p53 and cdk4. Lanes 1, 2, 3 and 4 contain protein samples from R2F, R2F (p53DD), R2F (cdk4^{R24C}) and R2F (p53DD/cdk4^{R24C}) fibroblasts, respective. β-actin was used as a loading control. FIG. 12b: Contribution of p53 and pRb pathways to T-oligo-induced SA-β-Gal activity. R2F fibroblasts and derived transductants were treated with diluent or 40 μM T-oligo for one week and then assayed for SA-β-Gal activity. FIG. 12c: Quantitative analysis of SA-β-Gal positive cells. Cells expressing SA-β-Gal activity were counted and presented as percentage of total cells in the cultures. Averages and standard deviations were calculated from 3 representative fields from each of 3 independent experiments.

FIG. 13 shows that exposure of human fibrosarcoma HT-1080 cells to T-oligo induces senescence. FIG. 13a: Exposure to T-oligo increases SA-β-Gal activity. HT-1080 cells were treated for 4 days with diluent alone or 40 μM T-oligo or the complementary control oligo, then stained and assayed for SA-β-Gal activity. FIG. 13b: Quantitative analysis of SA-β-Gal positive cells. Cells expressing SA-β-Gal activity were counted and presented as percentage of total cells in the cultures. Averages and standard deviations were calculated from 3 representative fields from each of 3 independent experiments. FIG. 13c: Effect of T-oligo on cell proliferation. Cells were treated for 4 days as in FIG. 12 and assayed for DNA synthesis by BrdU incorporation. FIG. 13d: Quantitative analysis of BrdU incorporation. Dark black nuclei indicate BrdU incorporated into nuclear DNA. BrdU positive cells were calculated and presented as percentage of total cells in the cultures. Averages and standard deviations were calculated from 3 representative fields from each of 3 independent experiments. FIG. 13e: Effect of T-oligo on pRb phosphorylation. Cells were treated as in FIG. 13a and were then collected for protein analysis by western blot using 30 μg total protein and probed for pRb-ser780*, ser795* and ser807/811* (pRb phosphorylated at serine 780, serine 795 and serine 807/811 respectively). Lanes D, T and C contain protein samples from cells treated with diluent, T-oligo and complementary oligo respectively. β-actin was used as a loading control.

FIG. 14 shows the persistent effect of T-oligo removal on the senescent phenotype in human fibrogarcoma HT-1080 cells. Parallel cultures were treated as described in FIG. 13a. Cells were then washed once with PBS and refed with complete medium without additional treatment for 24 hours or 48 hours. FIG. 14a: SA-β-Gal activity. Cells were stained for SA-β-Gal activity. FIG. 14b: Cell cycle arrest. BrdU incorporation was assayed. FIG. 14c: Phosphorylation and activation of pRb. Immunoblot analysis was performed as described in FIG. 13e.

FIG. 15 shows the effect of prolonged exposure to T-oligo on clonogenic capacity of human fibrosarcoma HT-1080 cells. Cells were treated with diluent, 40 μM T-oligo or complementary oligo for one week, and then assayed. FIG. 15a: Appearance of stained dishes. FIG. 15b: Quantification of clonogenic capacity. Colonies of triplicate cultures were counted and plotted as percentage of diluent treated control.

FIG. 16 shows the effect of T-oligo on mean telomere length (MTL) in human fibrosarcoma HT-1080 cells. Cells were treated as described in FIG. 13a. Lanes 1, 2, 3 contain genomic DNA from cells treated with diluent (D), T-oligo (T), or complementary oligo (C). Lanes 4 and 5 contain high (H) molecular and low (L) molecular weight standard telomeric DNA.

FIG. 17 shows that T-oligos and TRF^{DN} initiate DNA damage responses via the same pathway. The graphs show densitometric readings of the western blots, with diluent control set at 100%. FIG. 9f: Lane 1, diluent, GFP; lane 2, diluent TRF2DN; lane 3, 3AB, GFP; lane 4, 3AB, TRF2DN; lane 5, IQ, GFP; lane 6, IQ, TRF2DN.

FIG. 18 shows that the effect of T-oligos are not dependent on telomerase. FIG. 18a: FACS profiles from one representative experiment of three with the percentage and standard deviations of cells in each phase of the cell cycle was calculated from triplicate cultures of each condition. FIG. 18b: Western blots with an antibody specific for phospho-p95/Nbs1. Lanes 1, 2 and 3 contained protein from cells treated with diluent, 11mer-1 or 11mer-2, respectively. Control cells (3 hours) were irradiated with 10 Gy of IR (+), or were sham irradiated (−).

FIG. 19 shows that downregulating tankyrase protein levels blocks the response of T-oligos. The upper panel shows the densitometry readings and the lower panel shows the western blot.

FIG. 20 shows that T-oligos cause phosphorylation of p53 on serine 37. Western blot analysis was performed on normal neonatal cells using a antibody specific for p53 phosphoserine 37 after being treated with either diluent or 40 μM for the indicated times.

### DETAILED DESCRIPTION OF THE INVENTION

The present invention is based on the discovery that Mre11-mediated hydrolysis of the 3′ telomere overhang sequence initiates signaling cascades important for protective cellular responses to DNA damage, such as senescence, tanning and apoptosis. Not being bound by theory, we believe that DNA damage, such as UV irradiation, oxidative damage to DNA, or formation of carcinogen adducts to DNA, or age-associated telomere shortening destabilizes the telomere loop, exposing the 3′ overhang sequence comprising repeats of TTAGGG. Telomere-associated proteins then attach to the overhang in a sequence-dependent manner and serve as an “anchor” for the Mre11/p95/Rad50 complex. Mre11 then begins to hydrolyze the telomere overhang from the 3′ end, which leads to activation of the Rad50 ATPase. Activation of Rad50 leads to activation of tankyrase by phosphorylation, conformational change of some kind or other mechanism, which then activates ATM and possibly other kinases such as ATR. ATM then phosphorylates p95 and other DNA damage response effectors such as p53, ultimately leading to the biologic endpoints of cell cycle arrest, gene induction, apoptosis and/or senescence.

Based on the role of Mre11 and tankyrase in the proposed signaling pathway, activators of Mre11, tankyrase, the DNA damage pathway or MRN complex formation are expected to activate the DNA damage response pathway regardless of the presence of DNA damage or telomere loop disruption. This is illustrated in the Examples herein showing that telomere homolog oligonucleotides (T-oligos) serve as a substrate for Mre11 thereby leading to apoptosis, senescence or growth arrest in the absence of DNA damage or telomere loop disruption.

Similarly, inhibitors of Mre11, tankyrase, the DNA damage pathway or MRN complex formation are expected to inhibit the signal transduction pathway, even in the presence of DNA damage or telomere loop disruption. This is illustrated in the Examples herein showing that apoptosis and growth arrest are inhibited under conditions that cause DNA damage and telomere loop disruption by the following: (i) non-hydrolyzable T-oligos, which act as an antagonist of Mre11, (ii) RNAi-mediated reduction in Mre11 protein levels; and (iii) RNAi-mediated reduction in tankyrase protein levels.

Before the present products, compositions and methods are disclosed and described, it is to be understood-that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

Throughout this application, where patents or publications are referenced, the disclosures of these publications in their entireties are hereby incorporated by reference into this application in order to more fully describe the state of the art to which this invention pertains.

### 1. Definitions

As used herein, the term “activator” means anything that activates a protein or increases the activity of a protein.

As used herein, the term “administer” when used to describe the dosage of a modulator means a single dose or multiple doses of the agent.

As used herein, the term “analog”, when used in the context of a peptide or polypeptide, means a peptide or polypeptide comprising one or more non-standard amino acids or other structural variations from the conventional set of amino acids; and, when used in the context of an oligonucleotide, means an oligonucleotide comprising one or more internucleotide linkages other than phosphodiester internucleotide linkages.

As used herein, the term “antibody” means an antibody of classes IgG, IgM, IgA, IgD or IgE, or fragments or derivatives thereof, including Fab, F(ab′)₂, Fd, and single chain antibodies, diabodies, bispecific antibodies, bifunctional antibodies and derivatives thereof. The antibody may be a monoclonal antibody, polyclonal antibody, affinity purified antibody, or mixtures thereof which exhibits sufficient binding specificity to a desired epitope or a sequence derived therefrom. The antibody may also be a chimeric antibody. The antibody may be derivatized by the attachment of one or more chemical, peptide, or polypeptide moieties known in the art. The antibody may be conjugated with a chemical moiety.

As used herein, “apoptosis” refers to a form of cell death that includes, but is not limited to, progressive contraction of cell volume with the preservation of the integrity of cytoplasmic organelles; condensation of chromatin (i.e., nuclear condensation), as viewed by light or electron microscopy; and/or DNA cleavage into nucleosome-sized fragments, as determined by centrifuged sedimentation assays. Cell death occurs when the membrane integrity of the cell is lost (e.g., membrane blebbing) with engulfment of intact cell fragments (“apoptotic bodies”) by phagocytic cells.

As used herein, the term “cancer treatment” means any treatment for cancer known in the art including, but not limited to, chemotherapy and radiation therapy.

As used herein, the term “combination with” when used to describe administration of a modulator and an additional treatment means that the modulator may be administered prior to, together with, or after the additional treatment, or a combination thereof.

As used herein, the term “derivative”, when used in the context of a peptide or polypeptide, means a peptide or polypeptide different other than in primary structure (amino acids and amino acid analogs); and, when used in the context of an oligonucleotide, means an oligonucleotide different other than in the nucleotide sequence. By way of illustration, derivatives of a peptide or polypeptide may differ by being glycosylated, one form of post-translational modification. For example, peptides or polypeptides may exhibit glycosylation patterns due to expression in heterologous systems. If at least one biological activity is retained, then these peptides or polypeptides are derivatives according to the invention. Other derivatives include, but are not limited to, fusion peptides or fusion polypeptides having a covalently modified N- or C-terminus, PEGylated peptides or polypeptides, peptides or polypeptides associated with lipid moieties, alkylated peptides or polypeptides, peptides or polypeptides linked via an amino acid side-chain functional group to other peptides, polypeptides or chemicals, and additional modifications as would be understood in the art.

As used herein, the term “fragment”, when used in the context of a peptide or polypeptide, means any peptide or polypeptide fragment, preferably from about 5 to about 300 amino acids in length, more preferably from about 8 to about 50 amino acids in length; and, when used in the context of an oligonucleotide, means any oligonucleotide fragment, preferably from about 2 to about 250 nucleotides, more preferably from about 2 to about 20 nucleotides in length. Representative examples of peptide or polypeptide fragments are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids in length. Representative examples of oligonucleotide fragments are 2, 3, 4, 5, 6, 7, 8, 9, 10, 11, 12, 13, 14, 15, 16, 17, 18, 19 or 20 nucleotides in length.

As used herein, the term “homolog”, when used in the context of a peptide or polypeptide, means a peptide or polypeptide sharing a common evolutionary ancestor or having at least 50% identity thereto; and, when used in the context of an oligonucleotide, means an oligonucleotide sharing a common evolutionary ancestor or having at least 50% identity thereto.

As used herein, the term “inhibit′ when referring to the activity of a protein, means preventing, suppressing, repressing, or eliminating the activity of the enzyme.

As used herein, the term “treat” or “treating” when referring to protection of a mammal from a condition, means preventing, suppressing, repressing, or eliminating the condition. Preventing the condition involves administering a composition of the present invention to a mammal prior to onset of the condition. Suppressing the condition involves administering a composition of the present invention to a mammal after induction of the condition but before its clinical appearance. Repressing the condition involves administering a composition of the present invention to a mammal after clinical appearance of the condition such that the condition is reduced or prevented from worsening. Elimination the condition involves administering a composition of the present invention to a mammal after clinical appearance of the condition such that the mammal no longer suffers the condition.

As used herein, the term “variant”, when used in the context of a peptide or polypeptide, means a peptide or polypeptide that differs in amino acid sequence by the insertion, deletion, or conservative substitution of amino acids, but retain at least one biological activity; and, when used in the context of an oligonucleotide, means an oligonucleotide that differs in nucleotide sequence by the insertion, deletion, or substitution of nucleotides, but retain at least one biological activity. For purposes of the present invention, “biological activity” includes, but is not limited to, the ability to be bound by a specific antibody.

### 2. Modulators

### a. Modulator of Mre11

The present invention relates to a modulator of Mre11 activity. The modulator may induce or increase Mre11 activity. The modulator may also inhibit or reduce Mre11 activity. The modulator may be an artificially synthesized compound or a naturally occurring compound. The modulator may be a low molecular weight compound, oligonucleotide, polypeptide or peptide, or a fragment, analog, homolog, variant or derivative thereof.

An oligonucleotide modulator may be an oligonucleotide with at least about 50% to about 100% nucleotide sequence identity with (TTAGGG)ₙ, wherein n is from about 1 to about 333. The oligonucleotide may be of a form including, but not limited to, single-stranded, double-stranded, or a combination thereof The oligonucleotide preferably comprises a single-stranded 3′-end of from about 2 to about 2000 nucleotides, more preferably from about 2 to about 200 nucleotides. The oligonucleotide may also be an EST. Also specifically contemplated is an analog, derivative, fragment, homolog or variant of the oligonucleotide.

As shown in the Examples, certain oligonucleotides of the present invention caused the inhibition of proliferation and induction of apoptosis in cells, whereas other oligonucleotides of the present invention cause the inhibition of growth arrest and inhibition of apoptosis. The difference in the activity of the oligonucleotides was dependent on the number of 3′ hydrolyzable internucleotide linkages. By varying the number of 3′ hydrolyzable internucleotide bonds, the effect of the oligonucleotides was varied.

Not being bound by theory, we believe that the oligonucleotides are recognized by the MRN complex and serve as a substrate for the 3′-exonuclease Mre11. The corollary is that substrate oligonucleotides that comprise 3′-nonhydrolyzable internucleotide bonds act as antagonists or inhibitors of Mre11. Other factors determining the level of Mre11 activity include, but are not limited to, the total concentration of 3′-hydrolyzable internucleotide bonds, base sequence and G content.

An internucleotide bond is considered hydrolyzable for purposes of the present invention if (i) it is a phosphodiester linkage or an analog thereof that is hydrolyzable by Mre11 under physiological conditions, and (ii) all internucleotide bonds 3′ thereto are also hydrolyzable. An internucleotide bond is considered nonhydrolyzable for purposes of the present invention if it is not hydrolyzable by Mre11 under physiological conditions, regardless of the number of hydrolyzable internucleotide linkages that are 3′ thereto. Representative examples of nonhydrolyzable internucleotide linkages include, but are not limited to, phosphorothioate linkages and peptide nucleic acid linkages (PNA).

In one embodiment of the invention, the oligonucleotide comprises hydrolyzable internucleotide bonds. The oligonucleotide may comprise from about 1 to about 200 hydrolyzable internucleotide bonds. The oligonucleotide may also comprise nonhydrolyzable internucleotide bonds. The oligonucleotide may comprise from about 0 to about 199 nonhydrolyzable internucleotide bonds.

In another embodiment, the oligonucleotide comprises nonhydrolyzable bonds. The oligonucleotide may comprise from about 1 to about 200 nonhydrolyzable internucleotide bonds. The oligonucleotide may also comprise hydrolyzable internucleotide bonds. The oligonucleotide comprise from about 0 to about 5 hydrolyzable internucleotide bonds. Preferred oligonucleotides are T-oligos described herein and as described in co-pending U.S. patent application Ser. No. 10/122,630, filed Apr. 12, 2002, which is incorporated herein by reference.

### b. Modulator of Tankyrase

The present invention also relates to a modulator-of tankyrase activity. The modulator may induce tankyrase activity. The modulator may also inhibit tankyrase activity. The modulator may-be an artificially synthesized compound-or a naturally occurring compound. The modulator may be a low molecular weight compound, polypeptide or peptide, or a fragment, analog, homolog, variant or derivative thereof.

### c. Modulator of the DNA Damage Pathway

The present invention also relates to a modulator of the DNA damage pathway. The modulator may induce the DNA damage pathway. The modulator may also inhibit the DNA damage pathway. The modulator may be an artificially synthesized compound or a naturally occurring compound. The modulator may be a low molecular weight compound, polypeptide or peptide, or a fragment, analog, homolog, variant or derivative thereof.

### d. Modulator of MRN Complex Formation

The present invention also relates to a modulator of MRN complex formation. The modulator may induce formation of the MRN complex. The modulator may also inhibit formation of the MRN complex. The modulator may be an artificially synthesized compound or a naturally occurring compound. The modulator may be a low molecular weight compound, polypeptide or peptide, or a fragment, analog, homolog, variant or derivative thereof.

### 3. Composition

The present invention also relates to a composition comprising a modulator as described above. The composition may comprise an activator of Mre11. The composition may also comprise an activator of tankyrase. The composition may also comprise an inhibitor of Mre11. The composition may also comprise an inhibitor of tankyrase. The composition may also comprise more than one modulator of the present invention. The composition may also comprise one or more modulators together with an additional therapeutic.

In one embodiment of the present invention, the composition comprises an oligonucleotide of the present-invention. The oligononucleotide may comprise hydrolyzable internucleotide bonds or nonhydrolyzable internucleotide bonds, or a combination thereof. In a preferred embodiment, the oligonucleotide is an activator of Mre11. In another preferred embodiment, the oligonucleotide is an inhibitor of Mre11. As discussed above, the activity of the oligonucleotide may be adjusted to induce or inhibit Mre11 based on the total concentration of hydrolyzable internucleotide bonds.

### a. Formulation

Compositions of the present invention may be in the form of tablets or lozenges formulated in a conventional manner. For example, tablets and capsules for oral administration may contain conventional excipients including, but not limited to, binding agents, fillers, lubricants, disintegrants and wetting agents. Binding agents include, but are not limited to, syrup, accacia, gelatin, sorbitol, tragacanth, mucilage of starch and polyvinylpyrrolidone. Fillers include, but are not limited to, lactose, sugar, microcrystalline cellulose, maizestarch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate). Tablets may be coated according to methods well known in the art.

Compositions of the present invention may also be liquid formulations including, but not limited to, aqueous or oily suspensions, solutions, emulsions, syrups, and elixirs. The compositions may also be formulated as a dry product for constitution with water or other suitable vehicle before use. Such liquid preparations may contain additives including, but not limited to, suspending agents, emulsifying agents,_nonaqueous vehicles and preservatives. Suspending agent include, but are not limited to; sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid.

Compositions of the present invention may also be formulated as suppositories, which may contain suppository bases including, but not limited to, cocoa butter or glycerides. Compositions of the present invention may also be formulated for inhalation, which may be in a form including, but not limited to, a solution, suspension; or emulsion that may be administered as a dry powder or in the form of an aerosol using-a propellant, such as dichlorodifluoromiethane or trichlorofluoromethane. Compositions of the present invention may also be formulated transdermal formulations comprising aqueous or nonaqueous vehicles including, but not limited to, creams, ointments, lotions, pastes, medicated plaster, patch, or membrane.

Compositions of the present invention may also be formulated for-parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

Compositions of the present invention may also be formulated as a depot preparation, which may be administered by implantation or by intramuscular injection. The compositions may be formulated with suitable polymeric or hydrophobic materials (as an emulsion in an acceptable oil, for example), ion exchange resins, or as sparingly soluble derivatives (as a sparingly soluble salt, for example).

Compositions of the present invention may also be formulated as a liposome preparation. The liposome preparation can comprise liposomes which penetrate the cells of interest or the stratum comeum, and fuse with the cell membrane, resulting in delivery of the contents of the liposome into the cell. For example, liposomes such as those described in U.S. Pat. No. 5,077,211 of Yarosh, U.S. Pat. No. 4,621,023 of Redziniak et al. or U.S. Pat. No. 4,508,703 of Redziniak et al. can be used. The compositions of the invention intended to target skin conditions can be administered before, during, or after exposure of the skin of the mammal to UV or agents causing oxidative damage. Other suitable formulations can employ niosomes. Niosomes are lipid vesicles similar to liposomes, with membranes consisting largely-of non-ionic lipids, some forms of which are effective for transporting compounds across the stratum corneum.

### 4. Methods of Treatment

### a. Activator of Mre11, Tankyrase, the DNA Damage Pathway or MRN Complex Formation

The modulators of the present invention that induce or increase the activity of Mre11 , tankyrase, the DNA damage pathway or MRN complex formation may be used alone or in combination with other treatments to treat conditions associated with failure of growth arrest, apoptosis or proliferative senescence. Representative examples of such conditions include, but are not limited to, hyperproliferative diseases, such as cancer and the benign growth of cells beyond a normal range as, for example, keratinocytes in psoriasis or fibroblast hypertrophic scars and keloids, or certain subsets of lymphocytes in the case of certain autoimmune disorders. The forms of cancer to be treated by these methods are manifested in various forms and arising in various cell types and organs of the body, for example, cervical cancer, lymphoma, osteosarcoma, melanoma and other cancers arising in the skin, and leukemia. Also among the types of cancer cells to which the therapies are directed are breast, lung, liver, prostate, pancreatic, ovarian, bladder, uterine, colon, brain, esophagus, stomach, and thyroid. The modulators may also be used to inhibit tanning, to promote cellular differentiation and for immunosuppresion.

In one embodiment of the present invention, an oligonucleotide of the present invention comprising hydrolyzable internucleotide bonds is used to treat a condition associated with failure of growth arrest, apoptosis or proliferative senescence by administering the oligonucleotide to a patient in need of such treatment. The oligononucleotide may also comprise nonhydrolyzable internucleotide bonds. As discussed above, the activity of the oligonucleotide may be adjusted to induce growth arrest or apoptosis based on the total concentration of hydrolyzable internucleotide bonds. The oligonucleotide may be administered in combination with modulators of the present invention or other treatments.

In a preferred embodiment, the 6ligonucleotide is used to treat a cancer selected from the group consisting of cervical, lymphoma, osteosarcoma, melanoma, skin, leukemia, breast, lung, liver, prostate, pancreatic, ovarian, bladder, uterine, colon, brain, esophagus, stomach, and thyroid.

T-oligos are capable of blocking induction or elicitation of allergic contact hypersensitivity as effectively as UV irradiation in a mouse model, through upregulation of TNF-α and IL10, known mediators of immunosuppression. A topical or systemic activator of Mre11 may, therefore, replace steroid therapy, for example, in treatment of lymphocyte-mediated skin diseases, such as psoriasis or eczema as well as lymphocyte-mediated systemic diseases such as rheumatoid arthritis, multiple sclerosis, lupus erythematosis, and many other diseases.

### b. Inhibitor of Mre11, Tankyrase, the DNA Damage Pathway or MRN Complex Formation

The modulators of the present invention that inhibit or decrease the activity of Mre11, tankyrase, the DNA damage pathway or MRN complex formation may be used alone or in combination with other treatments to treat conditions associated with growth arrest, apoptosis or proliferative senescence. Representative examples of such conditions include, but are not limited to, exposure to UV radiation and side effects of cancer treatments on normal tissues, such as chemotherapy and radiation therapy, or promoting the tanning response in sun exposed normal skin. The modulators may also be used to inhibit cellular differentiation.

In another embodiment, an oligonucleotide of the present invention comprising nonhydrolyzable internucleotide bonds is used to treat a condition associated with growth arrest or apoptosis by administering the oligonucleotide to a patient in need of such treatment. The oligononucleotide may also comprise hydrolyzable internucleotide bonds. As discussed above, the activity of the oligonucleotide may be adjusted to inhibit growth arrest or inhibit apoptosis based on the total concentration of hydrolyzable internucleotide bonds. The oligonucleotide may be administered in combination with modulators of the present invention or other treatments.

In a preferred embodiment, the oligonucleotide is used to treat a condition selected from the group consisting of exposure to UV radiation and side effects of cancer treatments, such as chemotherapy and radiation therapy.

### c. Administration

Compositions of the present invention may be administered in any manner including, but not limited to, orally, parenterally, sublingually, transdermally, rectally; transmucosally, topically, via inhalation, via buccal administration, or combinations thereof. Parenteral administration includes, but is not limited to, intravenous, intraarterial, intraperitoneal, subcutaneous, intramuscular, intrathecal, and intraarticular.

### d. Dosage

A therapeutically effective amount of the composition required for use in therapy varies with the nature of the condition being treated, the length of time that activity is desired, and the age and the condition of the patient, and is ultimately determined by the attendant physician. In general, however, doses employed for adult human treatment typically are in the range of 0.001 mg/kg to about 200 mg/kg per day. The dose may be about 1 μg/kg to about 100 μg/kg per day. The desired dose may be conveniently administered in a single dose, or as multiple doses administered at appropriate intervals, for example as two, three, four or more subdoses per day. Multiple doses often are desired, or required.

The dosage of a modulator may be at any dosage including, but not limited to, about 1 μg/kg, 25 μg/kg, 50 μg/kg, 75 μg/kg, 100 μg/kg, 125 μg/kg, 150 μg/kg, 175 μg/k, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 mg/kg, 800 μg/kg, 825 μg/kg, 850 μg/kg, 875 μg/kg, 900 μg/kg, 925 μg/kg, 950 μg/kg, 975 μg/kg or 1 mg/kg.

### 5. Screening Methods

The present invention also relates to screening methods of identifying modulators of Mre11 activity. The present invention also relates to screening methods of identifying modulators of tankyrase activity. The present invention further relates to screening methods of identifying modulators of MRN complex formation. Furthermore, the present invention relates to screening methods of identifying modulators of the DNA damage pathway. The screening methods may be performed in a variety of formats including, but not limited to, in vitro, cell-based, genetic and in vivo assays.

Modulators of Mre11 or tankyrase may be identified by screening for substances that specifically bind to Mre11 or tankyrase, as the case may be. Specific binding substances may be identified in vitro by one of ordinary skill in the art using a number of standard techniques including, but not limited to, immunoprecipitation and affinity chromatography. Specific binding substances may also be identified using genetic screens by one of ordinary skill in the art using a number of standard techniques including, but not limited to, yeast two-hybrid and phage display. Specific binding substances may also be identified using high throughput screening methods including, but not limited to, attaching Mre11 or tankyrase to a solid substrate such as a chip (e.g., glass, plastic or silicon).

Modulators of Mre11 or tankyrase may also be identified by screening in vitro for substances that modulate the activity of Mre11 or tankyrase, as the case may be. Modulators may be identified by contacting Mre11 or tankyrase with a suspected modulator and determining whether the suspected modulator alters the activity of Mre11 or tankyrase, as the case may be. The activity of Mre11 may be determined by measuring the hydrolysis of a nucleic acid substrate of Mre11. Hydrolysis of a nucleic acid substrate may be measured by methods including, but not limited to, measuring UV absorbance and, preferably, gel analysis of labeled oligos or recovery of non-precipitatable nucleotide bases. The activity of tankyrase may be determined by measuring the phosphorylation of a peptide or polypeptide including, but not limited to, TRF1.

A modulator of MRN complex formation may be identified in vitro by combining Mre11, Rad50 and Nbs1 and determining the effects of candidate modulators on MRN complex formation compared to a control. Formation of the MRN complex may be measured using a number of standard techniques known to one of ordinary skill in the art including, but not limited to, centrifugation, coprecipitation and nondenaturing electrophoresis.

A modulator of Mre11 or tankyrase may be identified by screening for substances that modulate the activity of Mre11 or tankyrase in cell-based assays. A modulator of the DNA damage pathway may similarly be identified. Modulators may be identified by contacting cells with a suspected modulator and determining whether the suspected modulator alters the level of apoptosis, senescence, or phosphorylation of p53 or p95. The candidate modulator may be a substance that specifically binds to Mre11 or tankyrase, as discussed above. Modulation of apoptosis may be measured by methods including, but not limited to, measuring the size of the sub-G₀/G₁ peak in FACS analysis, TUNEL assay, DNA ladder assay, annexin assay, or ELISA assay. Modulation of senescence may be determined by measuring senescence-associated β-galactosidase activity or failure to increase cell yields or to phosphorylate pRb or to incorporate ³H-thymidine after mitogenic stimulation. Modulation of p53 activity may be determined by measuring phosphorylation of p53 at serine 15 or serine 37 by gel shift assay by p53 promoter driven CAT or luciferase construct read-out, or by induction of a p53-regulated gene product such as p21. Modulation of p95 activity may be determined by measuring phosphorylation of p95 at serine 343 by shift in the p95 band in a western blot analysis, or by FACS analysis to detect an S phase arrest. A modulator of Mre11 or tankyrase may also be identified by screening for substances that modulate in vivo tumorigenecity.

Any cells may be used with cell-based assays. Preferably, cells for use with the present invention include mammalian cells, more preferably human and non-human primate cells. Representative examples of suitable cells include, but are not limited to, primary (normal) human dermal fibroblasts, epidermal keratinocytes, melanocytes, and corresponding immortalized or transformed cell lines; and primary, immortalized or transformed murine cells lines. The amount of protein phosphorylation may be measured using techniques standard in the art including, but not limited to, colorimetery, luminometery, fluorimetery, and western blotting.

The conditions under which a suspected modulator is added to a cell, such as by mixing, are conditions in which the cell can undergo apoptosis or signaling if essentially no other regulatory compounds are present that would interfere with apoptosis or signaling. Effective conditions include, but are not limited to, appropriate medium, temperature, pH and oxygen conditions that permit cell growth. An appropriate medium is typically a solid or liquid medium comprising growth factors and assimilable carbon, nitrogen and phosphate sources, as well as appropriate salts, minerals, metals and other nutrients, such as vitamins, and includes an effective medium in which the cell can be cultured such that the cell can exhibit apoptosis or signaling. For example, for a mammalian cell, the media may comprise Dulbecco's modified Eagle's medium containing 10% fetal calf serum.

Cells may be cultured in a variety of containers including, but not limited to tissue culture flasks, test tubes, microtiter dishes, and petri plates. Culturing is carried out at a temperature, pH and carbon dioxide content appropriate for the cell. Such culturing conditions are also within the skill in the art.

Methods for adding a suspected modulator to the cell include electroporation, microinjection, cellular expression (i.e., using an expression system including naked nucleic acid molecules, recombinant virus, retrovirus expression vectors and adenovirus expression), adding the agent to the medium, use of ion pairing agents-and use of detergents for cell permeabilization.

Candidate modulators may be naturally-occurring molecules, such as carbohydrates, monosaccharides, oligosaccharides, polysaccharides, amino acids, peptides, oligopeptides, polypeptides, proteins, nucleosides, nucleotides, oligonucleotides, polynucleotides, including DNA and DNA fragments, RNA and RNA fragments and the like, lipids, retinoids, steroids, glycopeptides, glycoproteins, proteoglycans and the like; or analogs or derivatives of naturally-occurring molecules, such peptidomimetics and the like; and non-naturally occurring molecules, such as “small molecule” organic compounds. The term “small molecule organic compound” refers to organic compounds generally having a molecular weight less than about 1000, preferably less than-about 500.

Candidate modulators may be present within a library (i.e., a collection of compounds), which may be prepared or obtained by any means including, but not limited to, combinatorial chemistry techniques, fermentation methods, plant and cellular extraction procedures and the like. Methods for making combinatorial libraries are well-known in the art. See, for example, E. R. Felder, Chimia 1994, 48, 512-541; Gallop et al., J. Med. Chem. 1994, 37, 1233-1251; R. A. Houghten, Trends Genet. 1993, 9, 235-239; Houghten et al., Nature 1991, 354, 84-86; Lain et al., Nature 1991, 354, 82-84; Carell et al., Chem. Biol. 1995, 3, 171-183; Madden et al., Perspectives in Drug Discovery and Design 2, 269-282; Cwirla et al., Biochemistry 1990, 87, 6378-6382; Brenner et al., Proc. Natl. Acad. Sci. USA 1992, 89, 5381-5383; Gordon et al., J. Med. Chem. 1994, 37, 1385-1401; Lebl et al., Biopolymers 1995, 37 177-198; and references cited therein.

The present invention has multiple aspects, illustrated by the following non-limiting examples.

### EXAMPLES

### Example 1

### Oligonucleotides can Induce Apoptosis

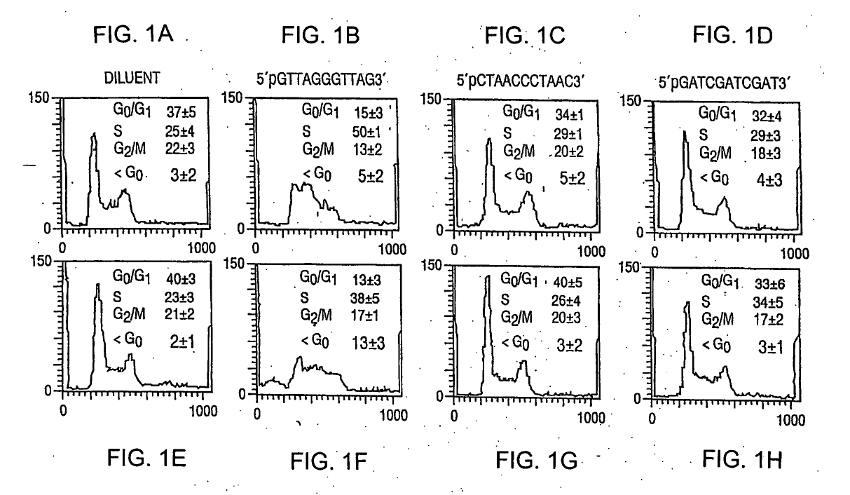

Oligonucleotides homologous to the telomere overhang repeat sequence (TTAGGG; SEQ ID NO: 1), sequence (11mer-1: pGTTAGGGTTAG; SEQ ID NO: 2), complementary to this sequence (11mer-2: pCTAACCCTAAC; SEQ ID NO: 3) and unrelated to the telomere sequence (11mer-3: pGATCGATCGAT; SEQ ID NO: 4) were added to cultures of Jurkat cells, a line of human T cells reported to undergo apoptosis in response to telomere disruption. Within′ 48 hours, 50% of the cells treated with 40 μM of SEQ ID NO: 5 had accumulated in the S phase, compared to 25-30% for control cells (p<0.0003, non-paired t-test; see FIGS. 1A-1D), and by 72 hours, 13% of these cells were apoptotic as determined by a sub-G₀/G₁ DNA content, compared to 2-3% of controls (p<0.007, non-paired t-test; see FIGS. 1E-1H). At 96 hours, 20±3% of the 11mer-1 treated cells were apoptotic compared with 3-5% of controls (p<0.0001, non-paired t-test). To exclude preferential uptake of the 11mer-1 as an explanation of its singular effects, Jurkat cells were treated with oligonucleotides labeled on the 3′ end with fluorescein phosphoramidite, then subjected to confocal-microscopy and FACS analysis. The fluorescence intensity of the cells was the same after all treatments at 4 hours and 24 hours. Western analysis showed an increase in p53 by 24 hours after addition of 11mer-1, but not 11mer-2 or 11mer -3, with a concomitant increase in the level of the E2F1 transcription factor, which is known to cooperate with p53 in induction of apoptosis and to induce a senescent phenotype in human fibroblasts in a p53-dependent manner as well as to regulate an S phase checkpoint.

### Example 2

### Phosphorothioate Version of the Telomere Overhang Homolog 11mer-1 Does Not Induce Apoptosis

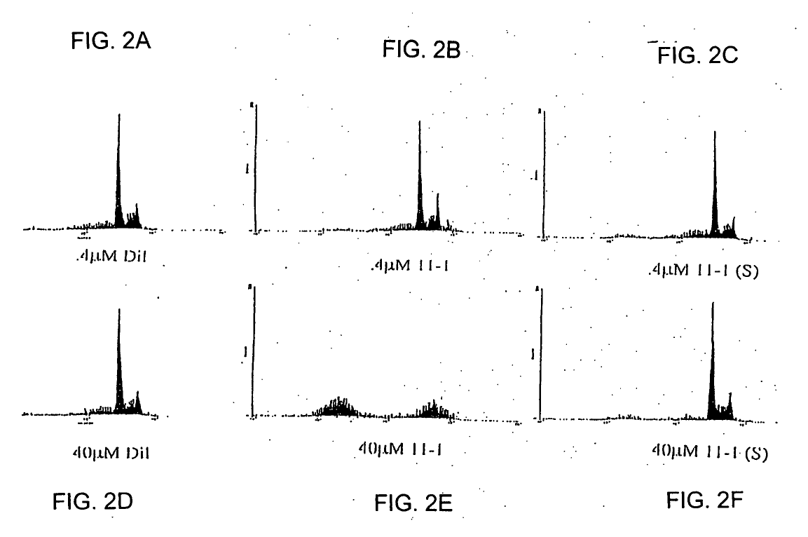

Cultures of Jurkat human T cells were treated with either diluent, 11mer-1 (SEQ ID NO: 1) or the phosphorothioate 11mer-1 (11mer-1-S) for 96 hours, then collected and processed for FACS analysis. Two concentrations of the oligonucleotides were tested, 0.4 μM (FIGS. 2A-2C) and 40 μM (FIGS. 2D-2F). At 0.4 μM, neither of the oligonucleotides affected the expected exponentially growing cell cycle profile of the Jurkat cells. At 40 μM, the 11-mer-1 induced extensive apoptosis, indicated by a sub-G₀/G₁ peak, while the 11mer-1-S had no effect.

### Example 3

### Phosphorothioate Version of 11mer-1 Blocks Induction of S-Phase Arrest by the Phosphate Backbone 11mer-1

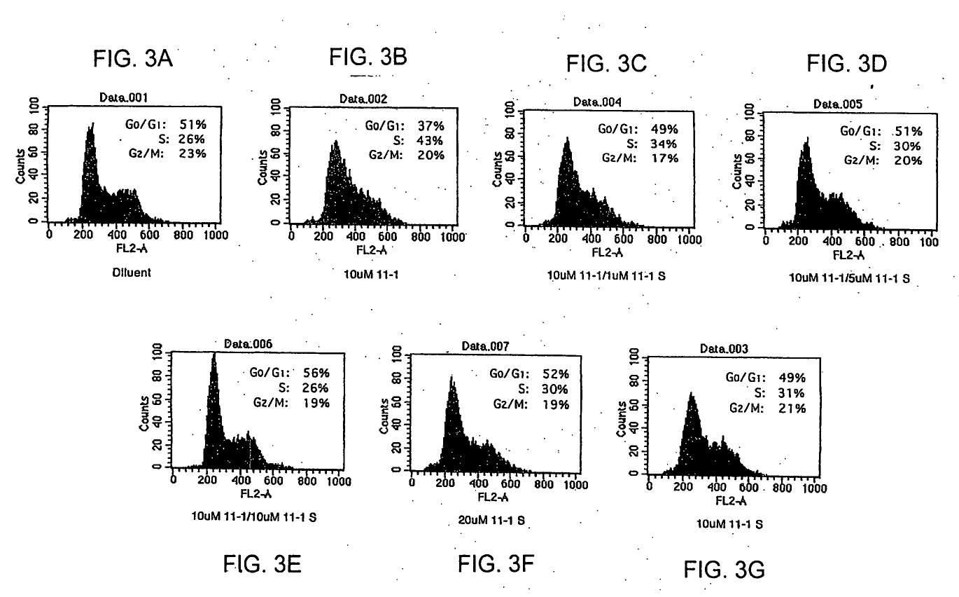

Cultures of a keratinocyte cell line (SSC12F, 100,000 cells/38 cm²) were treated for 48 hours with only the 11mer-1 (SEQ ID NO: 2) or with the 11mer-1 in the presence of increasing concentrations of the 11mer-1-S. As shown previously in Example 1, the 11mer-1 induced an S-phase arrest as demonstrated by FACS (Becton-Dickinson FacScan). Forty-three percent of the cells were in the S phase, compared to 26% of the control, diluent-treated cells. However, when increasing concentrations of the phosphorothioate 11mer-1 were also added to these cultures, fewer cells became arrested (FIGS. 3A-3G). Complete inhibition of this arrest was seen with a ratio of 11mer-1: 11mer-1-S of 2:1. The 11mer-1-S by itself did not induce the S-phase arrest.

### Example 4

### Phosphorothioate Forms of the Telomere Oligonucleotides Reduce Constitutive and UV-Induced Pigmentation and Do Not Stimulate Melanogenesis

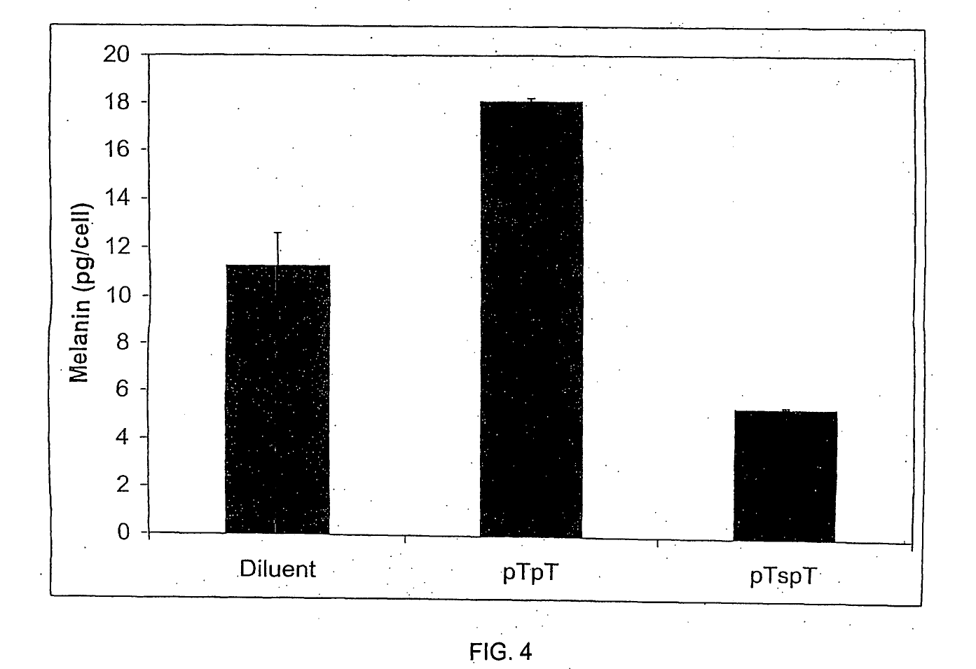

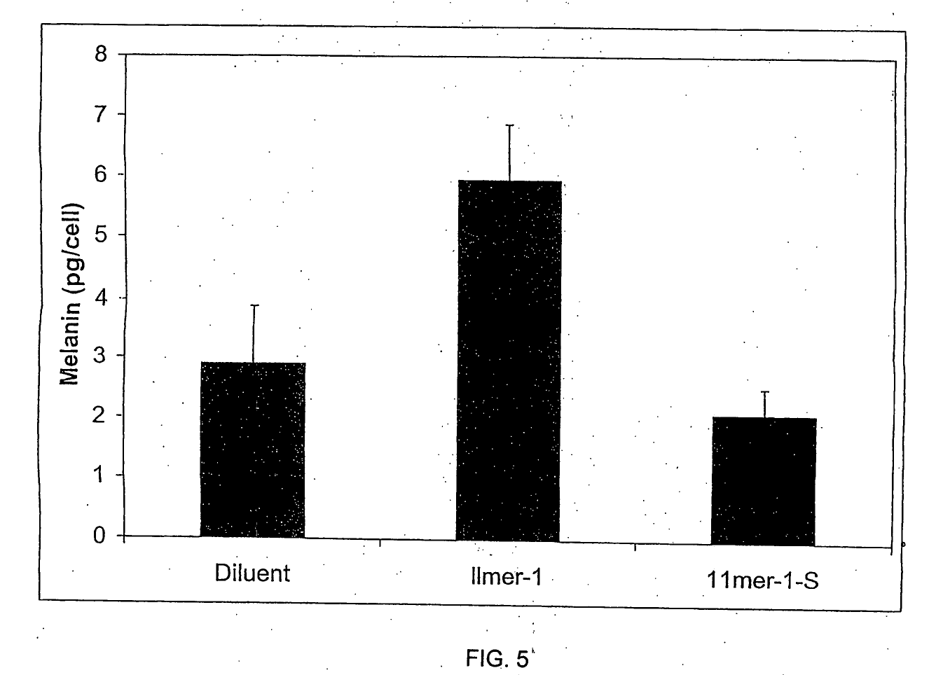

Cultures of S91 mouse melanoma cells (100,000 cells/38 cm²) were treated with 100 μM pTpT or phosphorothioate pTpT (pTspT) (FIG. 4) or 40 μM 11mer-1 or the phosphorothioate 11mer-1 (11mer-1-S) (FIG. 5) for 6 days and were then collected, counted and assayed for melanin content. While the pTpT and 11mer-1 (FIG. 4 and FIG. 5, respectively) stimulated melanogenesis in these cells, pTspT and 11mer-1-S did not (FIG. 4 and FIG. 5, respectively). Furthermore, both pTspT (FIG. 4) and 11mer-1-S (FIG. 5) reduced the constitutive pigmentation in these cells, suggesting that chronic exposure of this sequence during telomere repair/replication may provide a constant, low level signal for melanogenesis and this signal is blocked by pTspT and 11mer-1-S.

### Example 5

### Phosphorothioate pTspT Inhibits UV-Induced Melanogenesis

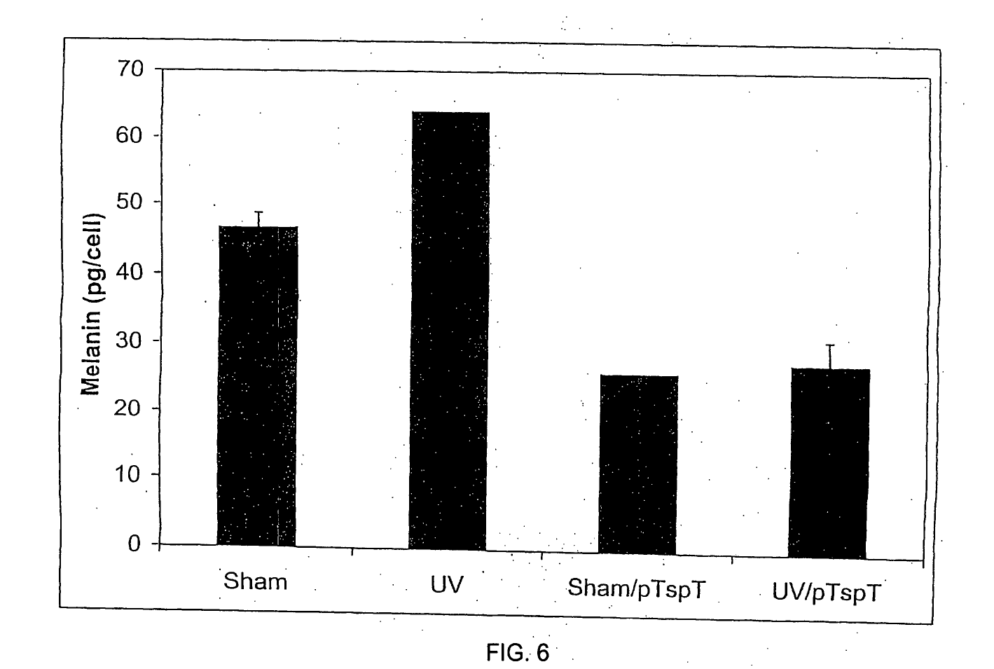

Duplicate cultures of S91 cells (100,000 cells/39 cm²) were either sham-irradiated or irradiated with 5 mJ/cm² solar-simulated light from a 1 kW xenon arc solar-simulator (XMN 1000-21, Optical Radiation, Azuza, Calif.) metered at 285±5 nm using a research radiometer (model IL1700A, International Light, Newburyport, Mass.). Two sham-irradiated plates were then supplemented with 100 μM pTspT and two irradiated cultures were similarly treated with pTspT. After one week, cells were collected, counted and analyzed for melanin content by dissolving the cell pellets in 1 N NaOH and measuring the optical density at 475 nm. UV irradiation resulted in a doubling of melanin content in these cells. However, this response was blocked by the addition of pTspT (FIG. 6). In addition, the constitutive pigmentation of these cells was reduced by the pTspT in the sham-irradiated cultures, similar to the data presented in FIGS. 4 and 5.

### Example 6

### Hydrolysis of the T-oligo is Necessary for Activity

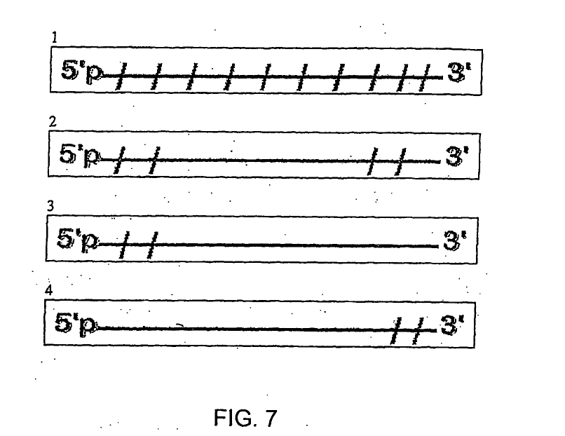

Oligonucleotides based on SEQ ID NO: 2 were synthesized. Oligonucleotide 1 was synthesized entirely with a phosphorothioate backbone. Oligonucleotide 2 had two phosphorothioate linkages on each end, with the other linkages in the middle being phosphodiester linkages. Oligonucleotide 3 had two phosphorothioate linkages on the 5′ end (5′ end blocked), with the rest of the linkages being phosphodiester linkages. Oligonucleotide 4 had two phosphorothioate linkages on the 3′ end (3′ end blocked), with the rest of the linkages being phosphodiester linkages. See FIG. 7.

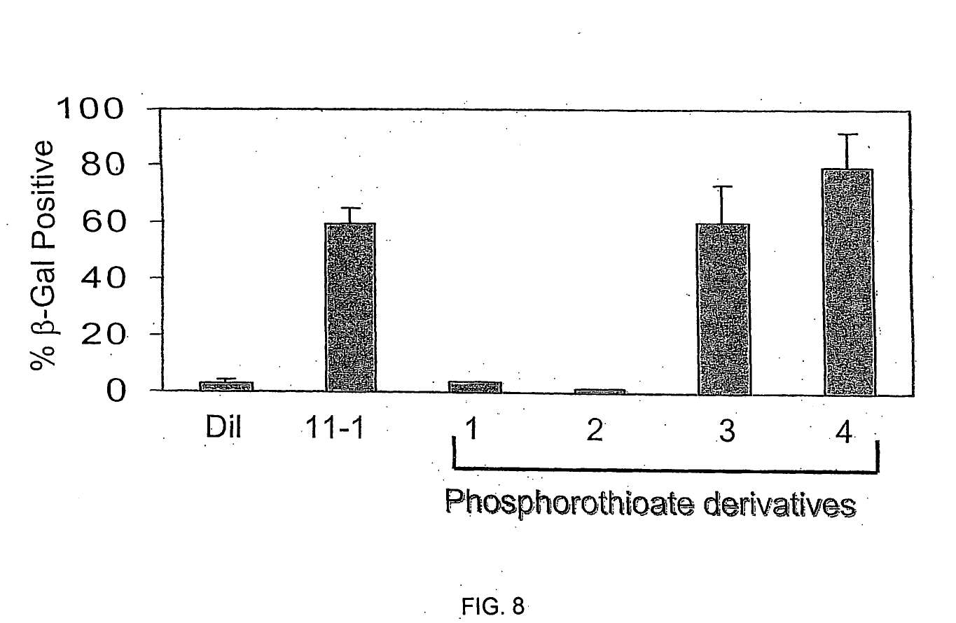

These oligonucleotides were added to cultures of normal neonatal fibroblasts. After 48 hours, cells were collected to be analyzed for p53 serine 15 phosphorylation and p95/Nbs1 phosphorylation by western blot. Other cultures were left in the presence of the oligonucleotides for one week and then the cells were stained for senescence-associated P-galactosidase activity (SA-β-Gal) β-galactosidase positive cells were scored and presented as a percent of total cells (FIG. 8).

Oligonucleotides with a nuclease-accessible 3′ terminus are the most effective at stimulating “early” responses such as p53 and p95/Nbs1 phosphorylation. However, oligonucleotides with a nuclease-accessible 5′ terminus can also induce the senescent phenotype after one week, but not the phosphorylation reactions at 48 hours, suggesting that 3′ to 5′ nuclease susceptibility is preferable for activity in inducing senescence.

### Example 7

### Downregulating Mre11 Protein Levels Blocks Response of T-Oligos

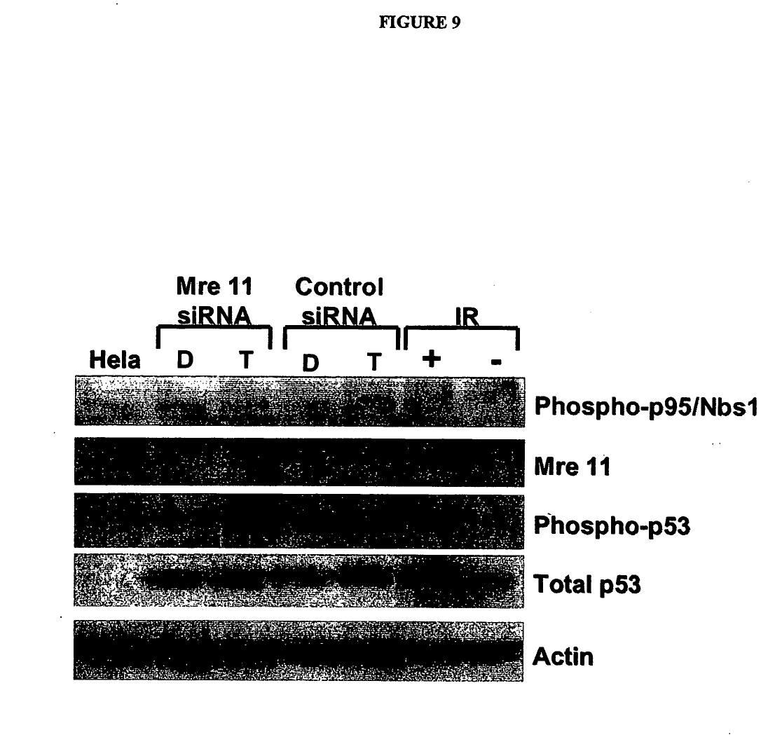

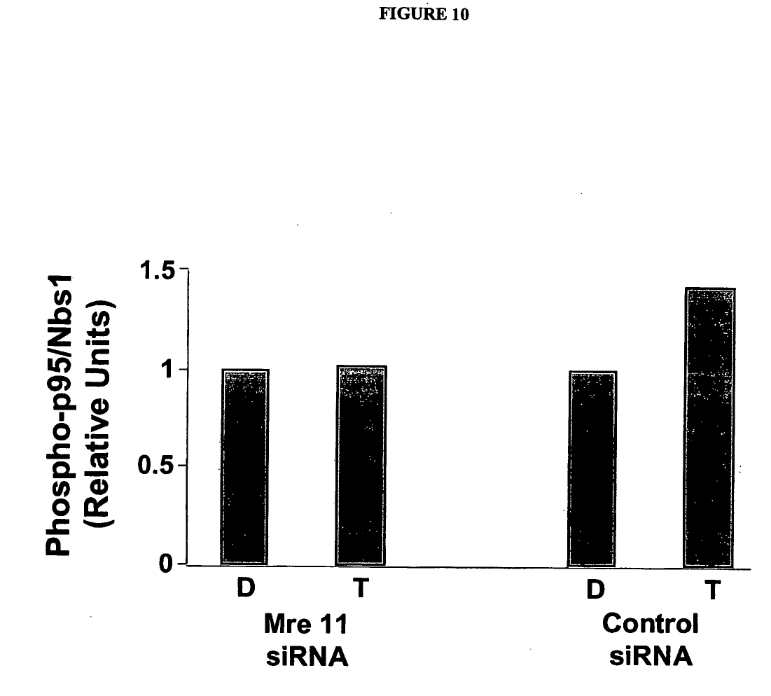

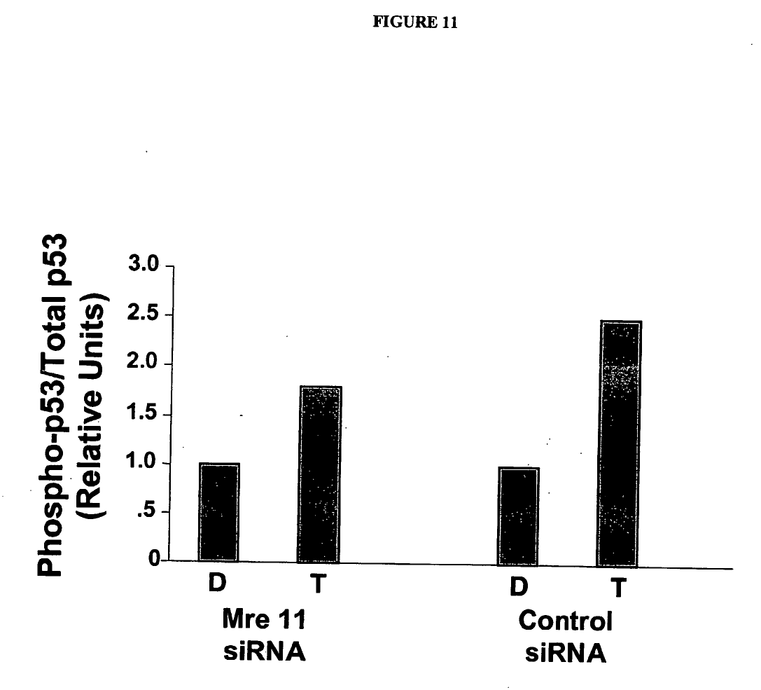

Normal human neonatal fibroblasts were treated with either 10 pmoles Mre11 siRNA or 10 pmoles control (no homology found in expressed human sequences). Cultures dishes were approximately 60% confluent on the day of siRNA transfection. Transfections were carried out using Lipofectamine 2000 (Invitrogen, Carlsbad, Calif.) following the protocol supplied by the manufacturer. The transfection cocktail was applied to the cells for 5 hours and then replaced with fresh medium alone. The next day, the transfection protocol was repeated. The following day, duplicate cultures were treated with the T-oligo or with diluent alone as a negative control. Cells were then collected 48 hours later and the protein analyzed by Western blot using antibodies specific for phospho-p95 serine 343 (Cell Signaling Technology, Beverly, Mass.), Mre11 (GeneTex, San Antonio, Tex.), phosphor-p53 serine 15 (Cell Signaling Technology) and total p53 (Oncogene, San Diego, Calif.) (FIG. 9). Hela cell lysate was used as a positive control for Mre11. Normal fibroblasts exposed to 10 Gy IR or sham irradiated and collected after one hour served as positive controls for p53 and p95/Nbs1 phosphorylation. The autoradiographs were analyzed by densitometry and the values for the T-oligo samples are expressed relative to the values for the diluent-treated samples (FIG. 10 and FIG. 11). After correcting for loading, it is apparent that cells with significantly reduced MRE 11 levels have a reduced phospho-p53 response to T-oligo and an absent phospho-p95/Nbs1 response.

### Example 8

### Inactivation of Both p53 and pRb Pathways is Necessary to Escape T-Oligo-Induced Senescence in R2F Fibroblasts

Oligonucleotides

Two DNA oligonucleotides were used, one homologous to the telomere overhang (T-oligo: pGTTAGGGTTAG; SEQ ID NO: 2) and one complementary thereto (CTAACCCTAAC; SEQ ID NO: 3), which was used as a negative control. These oligos were synthesized by the Midland Certified Reagent Company (Midland, Tex.). Oligonucleotides were prepared as previously described (Eller et al. [2003] Induction of a p95/Nbs1-mediated S phase checkpoint by telomere 3′ overhang specific DNA. Faseb J 17, 152-162).

Cell Source and Culture

R2F newborn dermal fibroblasts and derived p53DD, cdk4^{R14C} and p53DD/cdk4^{R24C} transductants (a generous gift from Dr. James G. Rheinwald of Harvard Medical School) lack a functional p53 pathway, pRb pathway, and both pathways respectively.

Senescence-Associated β-Galactosidase Staining

Cells were treated once with diluent alone, 40 μM T-oligo or 40 μM complementary oligo for 1 week without re-feeding. Cells were then fixed for 3-5 minutes in 2% formaldehyde/0.2% glutaraldehyde and incubated at 37° C. (ambient CO₂) overnight with fresh senescence-associated β-Gal (SA-β-Gal) stain solution, as described (Dimri et al. [1995] A biomarker that identifies senescent human cells in culture and in aging skin in vivo. Proc Natl Acad Sci USA 92, 9363-9367).

Western Blot Analysis and Antibodies

Western blot analysis was performed as previously described (Eller et al. [1996] DNA damage enhances melanogenesis. Proc Natl Acad Sci U S A 93, 1087-1092). The following antibodies were used: DO-1 (Ab-6) anti-p53 (Oncogene Research Products, Cambridge, Mass.), anti-phospho-p53 (ser 15) (Cell Signaling Technology Beverly, Mass.), anti-phospho-pRb (ser780, ser795, ser807/811) (Cell Signaling Technology Beverly, Mass.), anti-cdk4 (Cell Signaling Technology Beverly, Mass.) and anti-actin (Santa Cruz Biotechnology, Calif.).

Clonogenic Assay

Human fibrosarcoma cells were treated with diluent alone, 40 μM T-oligo or 40 μM complementary oligo for one week and were then trypsinized and counted. 300 cells were seeded into 60 mm culture dishes in triplicate and then incubated in complete medium for 2 weeks with medium changed twice per week. Subsequently, the cells were fixed for 5 min in 100% methanol. The methanol was then removed and the culture dishes were rinsed briefly with water. The colonies were stained for 10 min in 4% (w/v) methylene blue solution in PBS, washed once again with water, and then counted.

BrdU Incorporation Assay

HT-1080 fibrosarcoma cells cultured on Permanox chamber slides were treated with diluent, 40 μM T-oligo or 40 μM complementary oligo for 4 days and DNA synthesis was assayed using 5-bromo-2′-deoxy-uridine (BrdU) Labeling and Detection Kit II (Roche Molecular Biochemicals, Indianapolis, Ind.) following the protocol supplied by the manufacturer. Briefly, cells were labeled for 1 hour with BrdU, fixed and incubated with anti-BrdU monoclonal antibody. After incubation with anti-mouse-Ig-alkaline phosphatase, the color reaction was detected by light microscopy.

Telomere Length

HT-1080 fibrosarcoma cells were treated with diluent, 40 μM T-oligo or 40 μM complementary oligo for 4 days and then the genomic DNA was isolated using the DNeasy Tissue Kit (Qiagen, Valencia, Calif.). Telomere length was determined using the Telo TTAGGG Telomere Length Assay (Roche Molecular Biochemicals, Indianapolis, Ind.) following the protocol supplied by the manufacturer. Briefly, 1 μg of purified genomic DNA was digested with Hinf 1/Rsa1, the DNA fragments were separated on a 0.8% agarose gel and then transferred to a nylon membrane for Southern blotting, hybridized to a digoxigenin (DIG)-labeled probe specific for telomeric repeats and incubated with Anti-DIG-Alkaline Phosphatase. Terminal restriction fragments (TRF) were detected by chemiluminescence. The mean TRF length was calculated by scanning the exposed X-ray film with a densitometer and calculated as previously described (Harley et al. [1990] Telomeres shorten during ageing of human fibroblasts. Nature 345, 458-460).

Results

Senescent fibroblasts characteristically exhibit a large, flat morphology and an increase in senescence-associated β-galactosidase (SA-β-Gal) activity. Ectopic expression of TRF₂^{DN} disrupts the telomere loop structure and induces senescence in normal human fibroblasts by activating the p53 and pRb pathways. Blocking both the p53 and pRb pathways in human cells is required to prevent TRF₂^{DN} induced senescence.

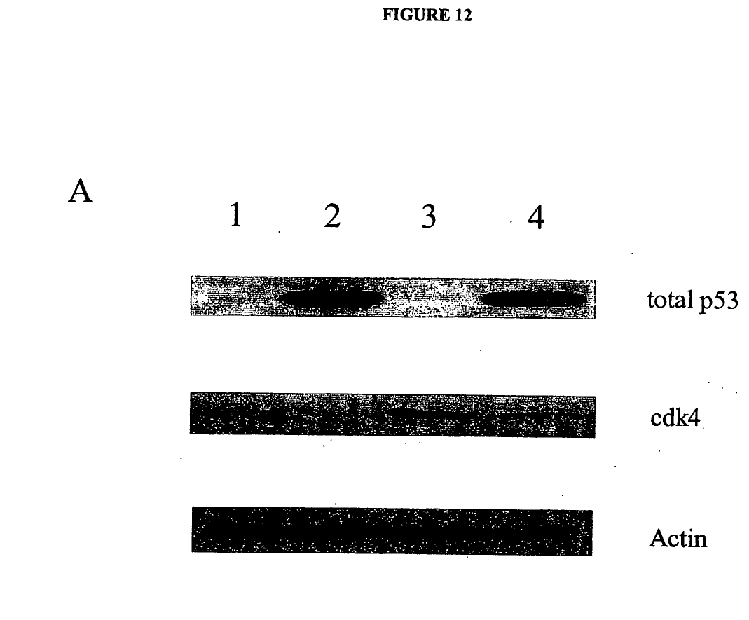

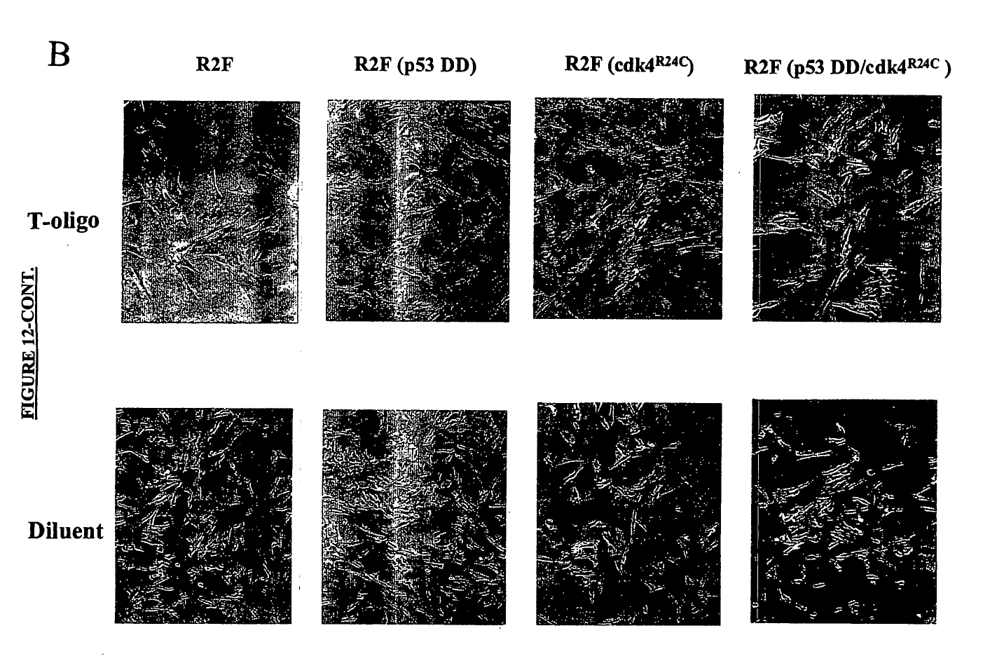

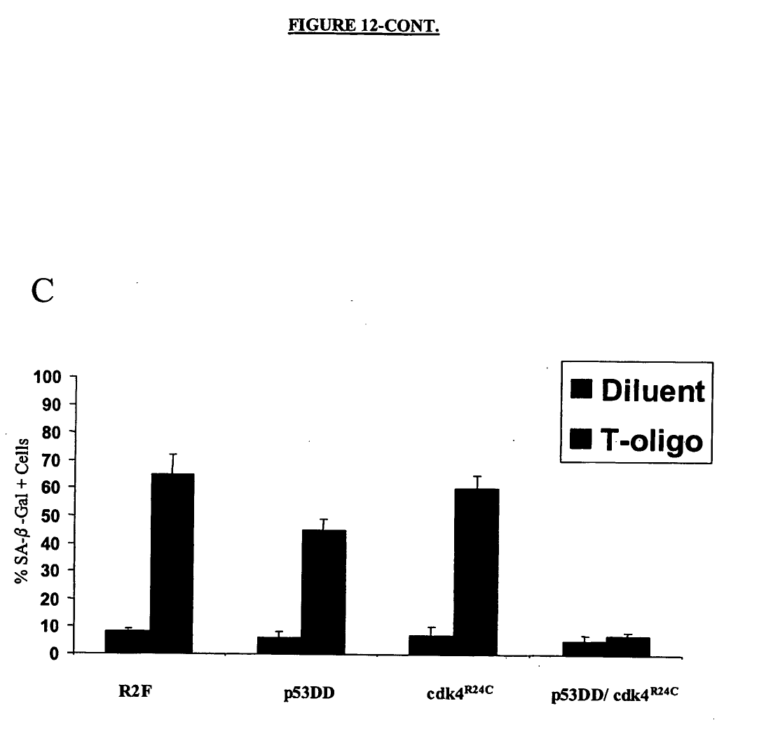

Cell lines engineered to lack the p53 pathway and/or pRb pathways were used to analyze the signaling pathways involved in T-oligo-induced senescence. Inactivation of the p53 pathway was achieved through ectopic expression of a dominant negative mutant p53 (p53DD) which lacks the transcriptional transactivation domain of p53 and binds and inactivates endogenous wild-type p53 protein. p21/SDI1 protein, a transcriptional target of p53, is below the level of detection in R2F fibroblasts transduced to expressed p53 DD (data not shown). The disruption of the pRb pathway was achieved through ectopic expression of a p16-insensitive mutant cdk4 (cdk4^{R24C}) unable to bind p16, thus abolishing its control of the pRb protein. The suppression of both pathways was achieved through ectopic expression of both mutants (p53DD/cdk4^{R24C}). Expression of p53DD and cdk4^{R24C} was confirmed by Western blot showing the overexpression of p53 and cdk4 proteins respectively (FIG. 12a), consistent with a previous report in which human keratinocytes were transduced with these mutants.

Cells were treated with either diluent or 40 μM T-oligo for 1 week and then assessed for SA-β-Gal activity. The normal neonatal foreskin fibroblast parental line (R2F) was used as a positive control. As expected, T-oligo-treated R2F fibroblasts exhibited a large, spread morphology and an increase in SA-β-Gal, activity as compared with diluent-treated control cells (65±7% and 8±1% SA-β-Gal positive cells, respectively, p<0.01) (FIG. 12b,c). Similarly, in p53DD R2F fibroblasts, one week exposure to T-oligo induced a large, spread morphology and an increase in SA-β-Gal activity as compared with diluent-treated cells (45±4% and 6±2% SA-β-Gal positive cells, respectively, p<0.01) (FIG. 12b,c), indicating that inactivation of the p53 pathway alone is not sufficient to suppress T-oligo-induced senescence. T-oligo also induced a senescent phenotype in cdk4^{R24C} R2F fibroblasts as compared with diluent-treated cells (60±5% and 7±3% SA-β-Gal positive cells, respectively, p<0.01) (FIG. 12b,c), indicating that the compromise of the pRb pathway alone is also not sufficient to suppress T-oligo induced senescence. However, when R2F fibroblasts were transduced to express both p53DD and cdk4^{R24C}, T-oligo was unable to induce a senescent phenotype as compared with diluent-treated cells (7±1% and 5±2% SA-β-Gal positive cells, respectively, p>0.05) (FIG. 12b,c), indicating that compromise of both the p53 and the pRb pathways is necessary to fully suppress T-oligo-induced senescence in human fibroblasts. Therefore, T-oligo-induced senescence has the same requirements as replicative senescence following serial passage or senescence induced by TRF2^{DN}.

### Example 9

### Inactivation of Both p53 and pRb Pathways is Necessary to Escape T-Oligo-Induced Senescence in HT-1080 Cells

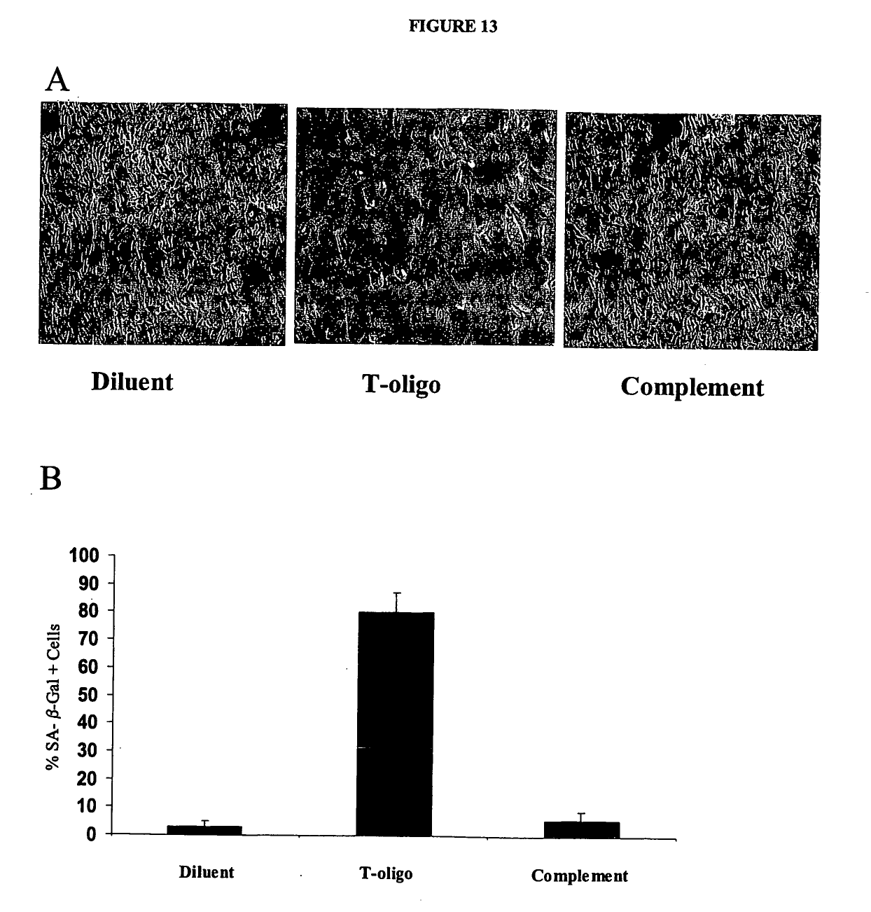

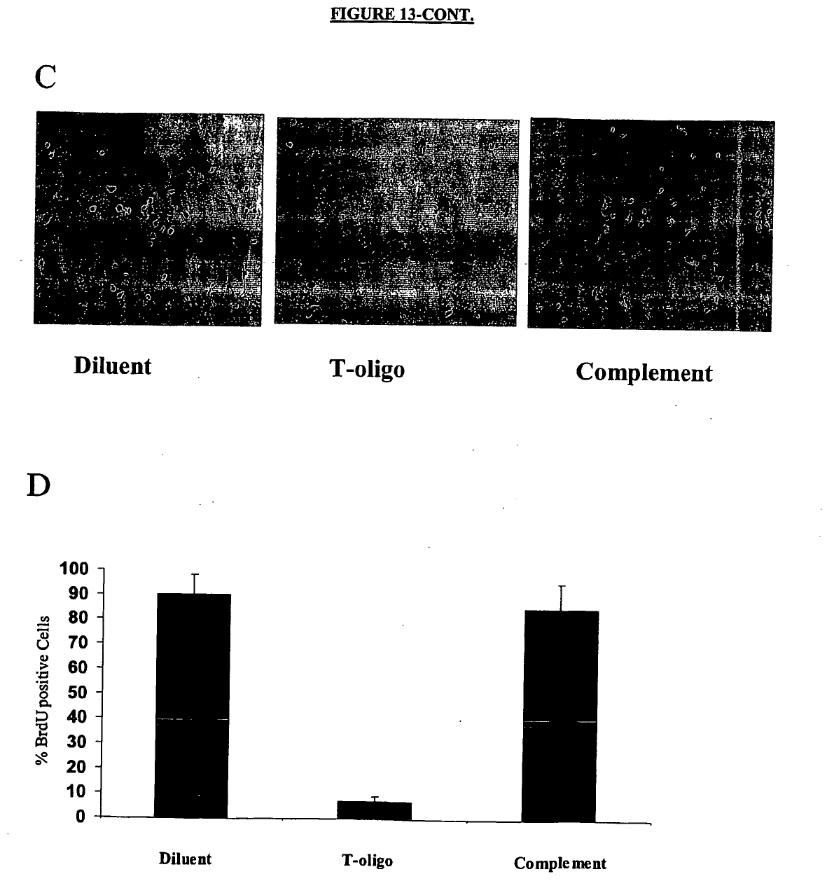

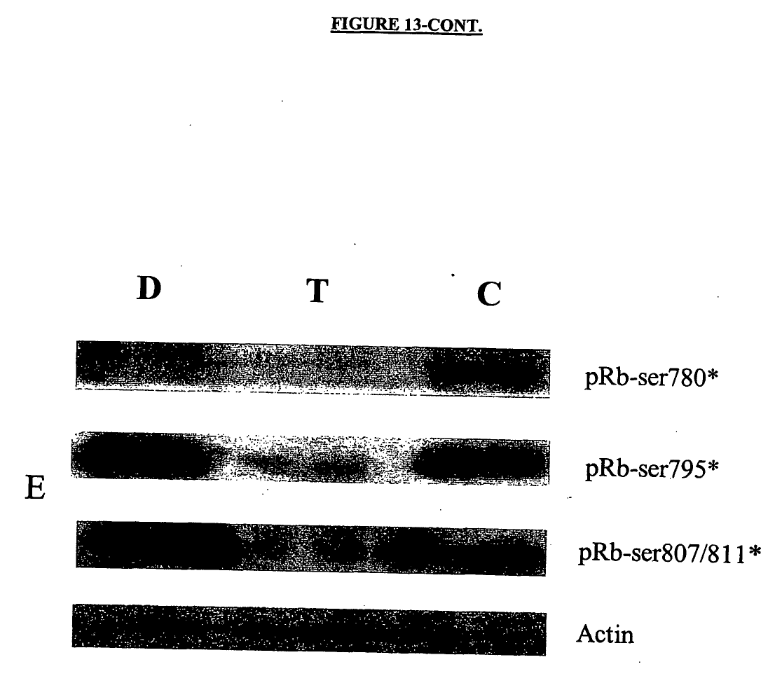

TRF2^{DN} has been reported to induce a senescent phenotype in human fibrosarcoma HT-1080 cells. To determine whether exposure to the telomere 3′ overhang DNA (T-oligo) also induces senescence in these cells, HT-1080 cells (American Type Culture Collection; Manassas, Va.) were treated with either diluent alone, T-oligo or the complementary oligo as a control, for 4 days and then assessed for SA-β-Gal activity. Only T-oligo-treated cells exhibited spread morphology and an increase in SA-β-Gal activity (FIG. 13a). T-oligo treated cultures contained many more SA-β-Gal positive cells than cultures treated with diluent or complementary control oligo (80±7%, 3±2% and 6±3%, respectively, p<0.01) (FIG. 13b). Also, only T-oligo-treated cells and not diluent or control oligo-treated cells were not proliferating as shown by pronounced reduction of BrdU incorporation (7±2%, 90±8% and 85±10%, respectively, p<0.01) (FIG. 13c,d).

### Example 10

### Telomere Oligonucleotides Prevent Phosphorylation of pRb

HT-1080 cells are known to have functional pRb, but the p53 pathway is deficient as a result of being p16 deficient. We next examined whether T-oligo treatment activates pRb by preventing its phosphorylation in HT-1080 cells. Western blot analysis revealed that there was a striking and selective reduction of pRb phosphorylation on serine 780, serine 795 and serine 807/811 in response to T-oligo (FIG. 13e). Interestingly, in tumors deficient in p16, pRb is often intact and functional. In these cells, the deregulation of cdk4 results in pRb hyperphosphorylation and leads to unrestricted cell growth and tumor formation. Cdk4, but not cdk2, activation phosphorylates pRb very efficiently on serine 780 and serine 795. The findings thus suggest that T-oligo inhibits cdk4 activity in the absence of p16, presumably through the induction of other INK4 family members, indicating the non-essential role of p16 in the complex network of pRb regulation and also suggesting that pRb can not be simply viewed as an absolute downstream effector.

### Example 11

### The Effects of Telomere oligonucleotides are not Reversible

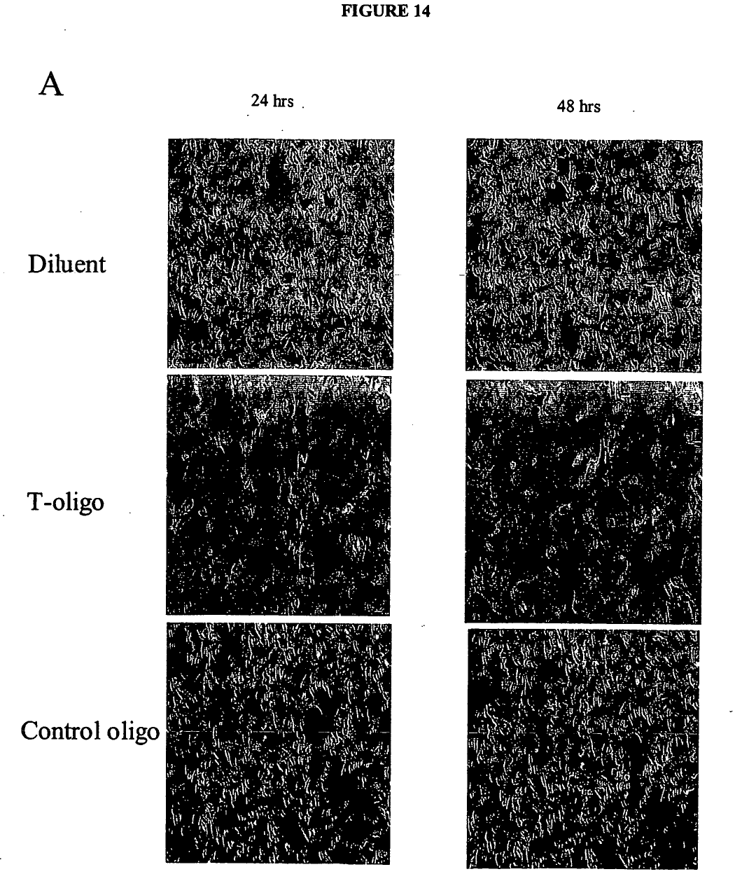

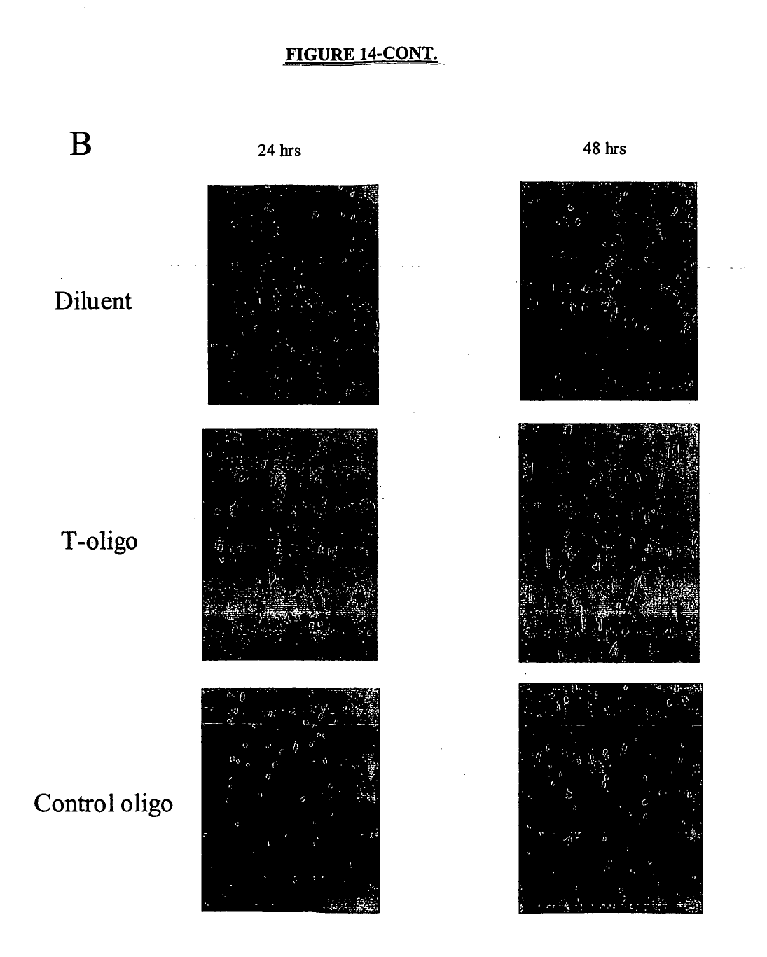

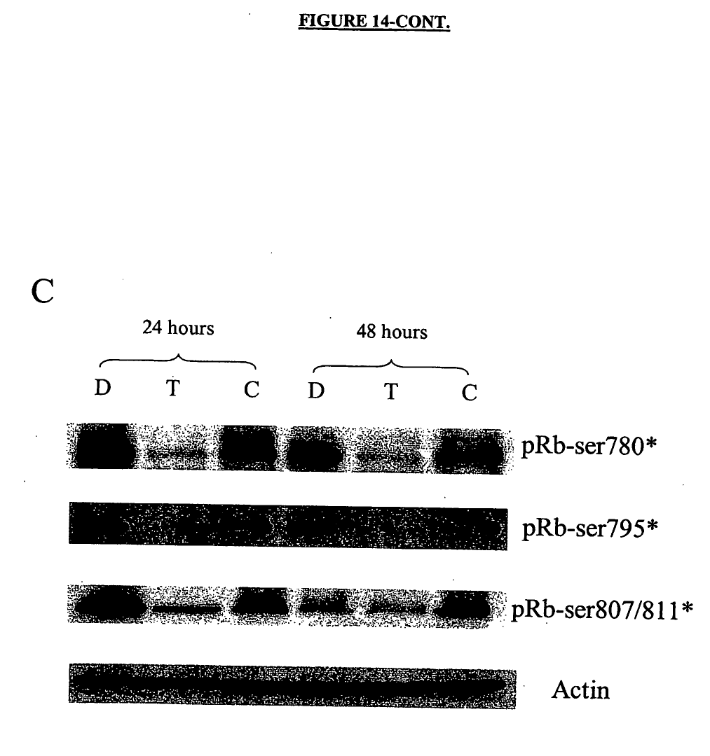

In order to test whether the removal of T-oligo would reverse the senescent phenotype of fibrosarcoma cells, parallel cultures of HT-1080 cells were treated for 4 days with diluent or 40 μM T-oligo or 40 μM complementary control oligo. Cells were then given fresh complete media without further oligonucleotide treatment. After 1 and 2 days, T-oligo pretreated cells still exhibited an enlarged morphology and an increase in SA-β-Gal activity (FIG. 14a) and did not resume DNA synthesis (FIG. 14b). Western analysis also showed that the pRb proteins were sustained in an active, inhibitory state in T-oligo pretreated cells (FIG. 14c).

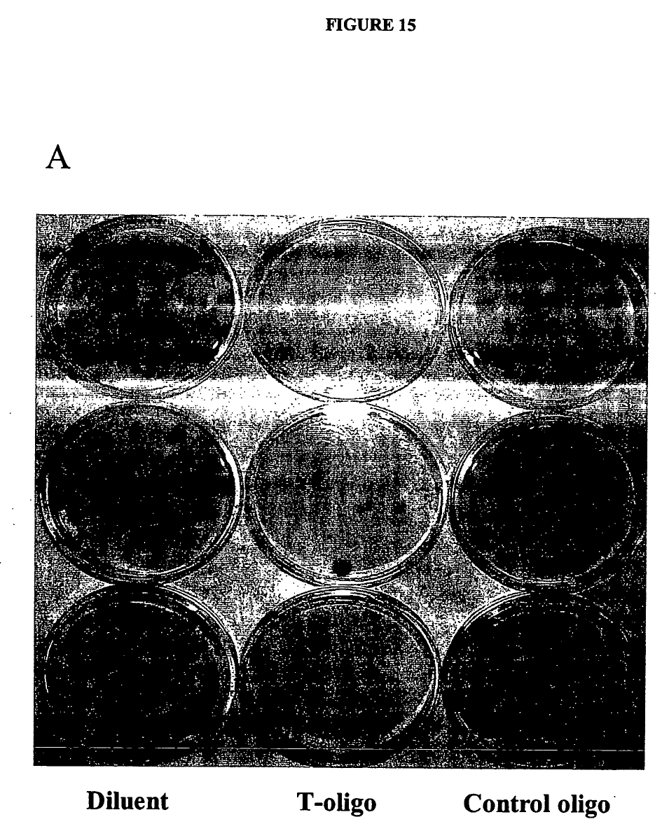

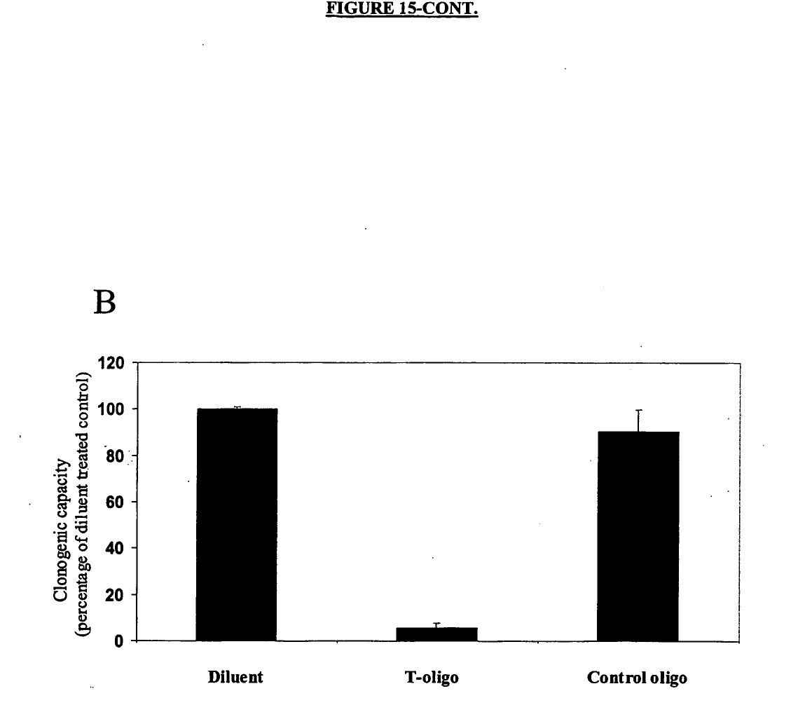

To determine the long-term effect of T-oligo treatment on cell growth, HT-1080 human fibrosarcoma cells were treated with either diluent alone, 40 μM T-oligo or 40 μM complementary control oligo for one week and then an equal number of cells were replated and medium was changed twice per week for 2 weeks with no further treatment, and then stained with methylene blue (FIG. 15a). Compared with complementary oligo-treated cells (90.5±9.4% of diluent treated control), the clonogenic. capacity of cells pretreated with T-oligo was almost completely suppressed (5.7±1.9% of diluent-treated control, p<0.01) (FIG. 15b). These data indicate that T-oligo induced senescence in this malignant cell line is not reversible.

### Example 12

### The Affect of Telomere Oligonucleotides on Mean Telomere Lengths

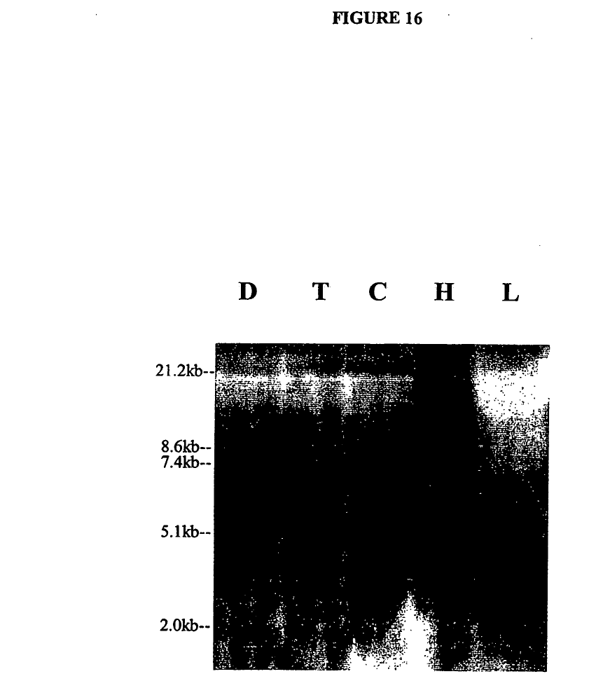

To determine the affect of T-oligos on the mean telomere length (MTL) in HT-1080 cells, cells were analyzed after treatment with T-oligo for 4 days which corresponded to the time that the senescent phenotype was readily observed. T-oligo did not alter MTL (5.56 kb) as compared with diluent-treated (5.61 kb) or complementary oligo treated controls (5.51 kb) (FIG. 16). The less than 100 bp difference in calculated MTL is within the range of experimental variation and is not significant. This is consistent with the observation that treatment of fibroblasts with T-oligo for up to 1 week does not result in degradation of the telomere 3′ overhang, as is observed following telomere disruption by TRF2^{DN} (data not shown). Because disruption of the telomere loop is known to cause rapid shortening of MTL and digestion of the 3′ overhang, the fact that T-oligo initiates similar or identical signaling without affecting MTL or causing digestion of the 3′ overhang indicates that the T-oligo mimics the exposure of the 3′ overhang sequence in the absence of telomere loop disruption, i.e., in the absence of DNA damage.

### Example 13

### PARP Activity is Required for T-oligo Responses

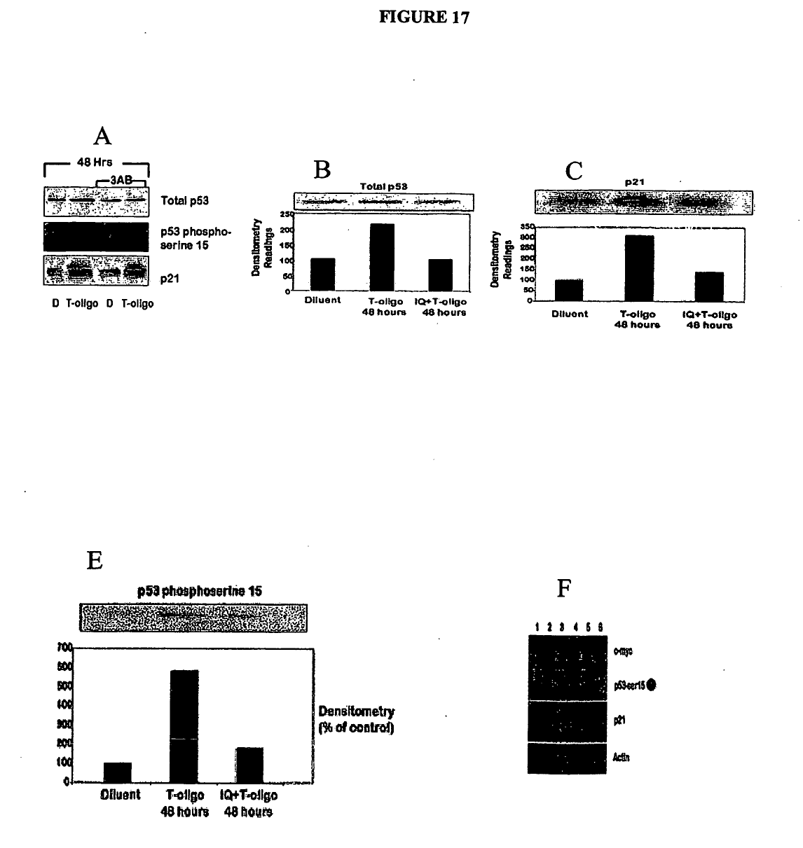

To investigate the role of PARPs in responses to T-oligo, fibroblasts were pretreated with one of two different PARP inhibitors, 3-aminobenzamide (3AB, 2.5 mM) or 1,5-dihydroxyquinoline (IQ, 100 μM) for 2 hours before addition of 40 μM T-oligo or an equal amount of diluent as a control. An additional dose of each inhibitor was given to the cells 4 hours after addition of the T-oligo or diluent (D). Fibroblasts were treated with 3AB and T-oligo, then collected 48 hours later for western blot. T-oligo-induced upregulation of total p53, p21, phosphorylation of p53 serine 15 (indicating p53 activation) were all reduced in the presence of 3AB (FIG. 17A).

Fibroblasts pretreated with IQ similarly showed reduced induction of total p53 and p53 phosphorylated on serine 15 at 16, 20 and 24 hours-after addition of T-oligo (data not shown). The effect of IQ on blocking T-oligo-mediated inductions of total p53, p53 phosphoserine 15, and p21 persisted through 48 hours after addition of T-oligo. These data demonstrate that the p53 responses to T-oligo require upstream PARP activity.

### Example 14

### PARP Inhibitors Prevent P53 Activation and Induction by TRF2^{DN}

Neonatal fibroblasts were treated with AdTRF2DN or AdGFP as a negative control. Two hours before infection, cells were treated with either diluent 3AB (2.5 mM) or IQ (100 μM). After 3 days cells were collected for western analysis for the c-myc-tagged TRF2DN (to confirm infection), p53 serine 15 phosphorylation and p21 induction. Comparing lane 2 to lanes 4 and 6 of FIG. 17F indicates that both 3AB and IQ reduced p53 phosphorylation and p21 induction in response to TRF2^{DN}.

### Example 15

### Effects of T-Oligos are not Dependent on Telomerase

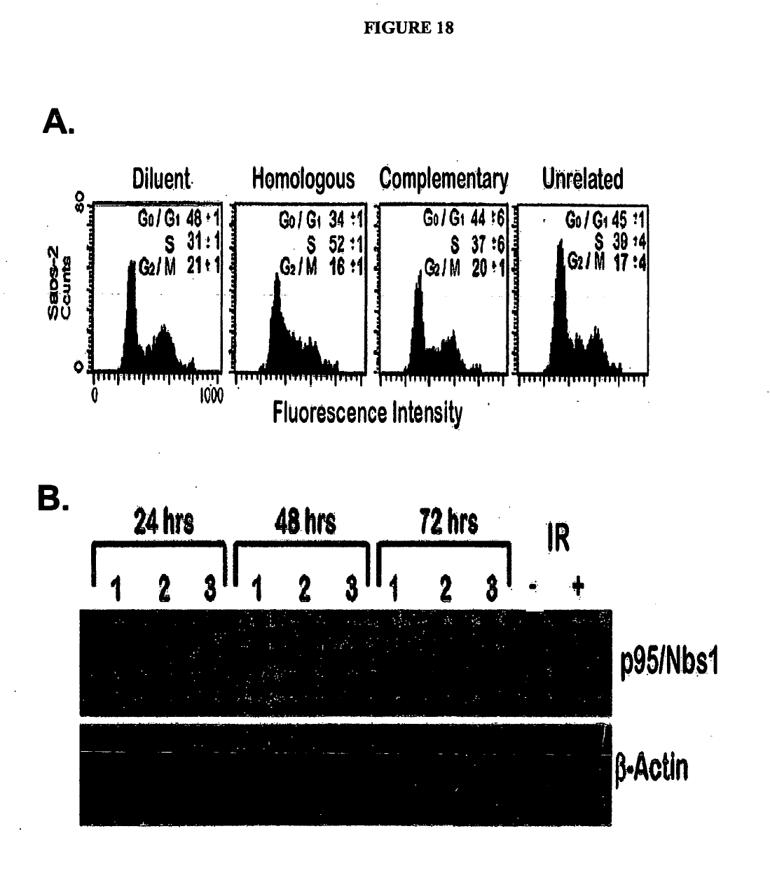

Saos-2 cells are an osteosarcoma cell line that is reportedly telomerase negative and maintain telomeres by the ALT pathway. Saos-2 cell lines were treated with either diluent or 40 μM of the indicated oligonucleotide and cells were collected after 48 hours for FACS analysis. Only the homologous nucleotide causes an S phase arrest of the cells (FIG. 18a). Furthermore, the telomere overhang oligonucleotide, as well as by IR, induced phosphorylation of p95/Nbs1 (FIG. 18b). The results that the effect of the T-oligo in the telomerase negative cells is identical to the response in telomerase positive malignant cell lines.

### Example 16

### Downregulating PARP Tankyrase Protein Levels Blocks Response of T-Oligos

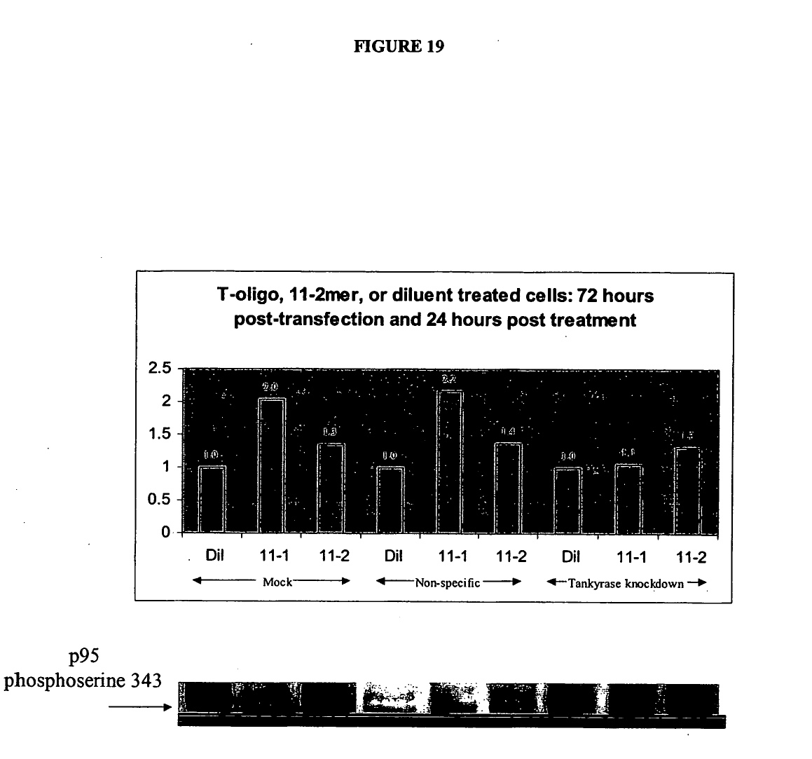

Paired cultures of human fibroblasts were treated once with tankyrase siRNA, with a non-specific siRNA (control) or were mock transfected as a second control. Two days later, when the tankyrase levels in tankyrase siRNA-treated cells was markedly reduced, the cultures were supplemented with 11-mer-1 (PGTTAGGGTTAG; SEQ ID NO: 2) or the complementary sequence 11-mer-2. After an additional 24 hours, cells were collected and processed for western blotting using an antibody specific for p95 phosphorylated at serine 343, indicating p95 modification by activated ATM kinase. The film was then subjected to densitometry and the diluent control for each group of cells was set at 1.0 in arbitrary units (FIG. 19). As expected, in cells with normal tankyrase levels the T-oligo treated cells had twice the amount of phosphorylated p95, while the control oligo-treated or diluent-treated cells had only a 30-40% increase. However, in the tankyrase knockdown group, the 11-mer-1 treated cells showed no increase in p95 phosphorylation (a level of 1.1 versus 1.0 and 1.3 for the controls). These data indicate that tankyrase, the telomere-associated PARP, is necessary to transduce the T-oligo signal that leads to ATM activation and subsequent modification (phosphorylation) of p95, thereby causing S-phase arrest of treated cells (Eller et al., FASEB J 2003).

### Example 17

### T-oligo Causes Non-ATM-Mediated Phosphorylation of p53

Normal neonatal fibroblasts were treated with either diluent or 40 μM (11mer-1) for 4, 6, 8, 19, 24 and 48 hours and then collected for Western blot analysis using an antibody specific for p53 phosphoserine 37. Sham and IR-irradiated (10 Gy) fibroblasts were used used as negative and positive controls, respectively. Increased band intensity in the western blot, corresponding to p53 serine 37, is detected as early as 8 hours and is very prominent at 48 hours in T-oligo (T)-treated vs diluent (D)-treated samples.”

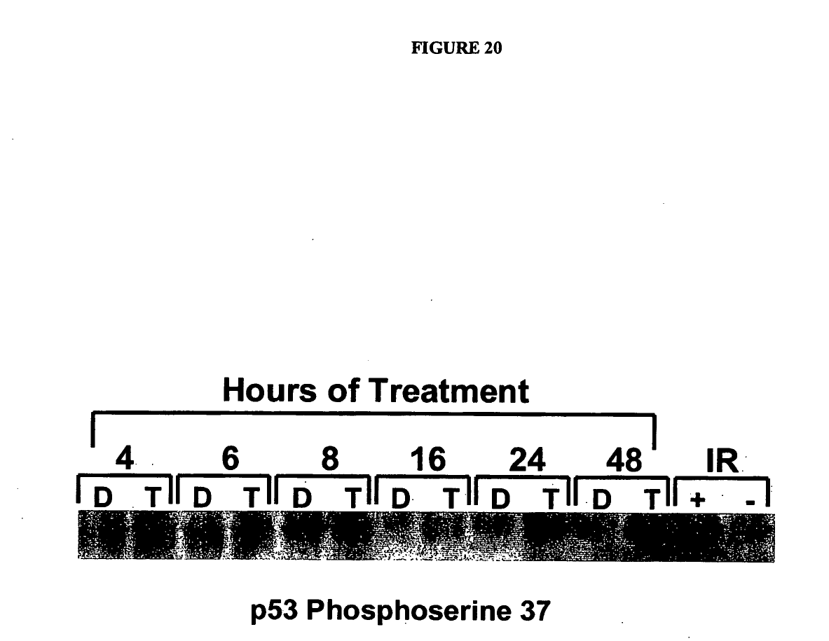

As shown above, T-oligo causes phosphorylation of p53 on serine 15. Phosphorylation of p53 at serine 15 is mediated by ATM. FIG. 20 indicates that T-oligos also cause phosphorylation of p53 on serine 37. Phosphorylation of p53 at serine 37 is mediated by either the ATM-related (ATR) kinase or the DNA-PK kinase, but is not known to be mediated by ATM. Demonstration of p53 serine 37 is thus another marker of pathway activation and one or both of these kinases are downstream targets of Mre11 activation. Moreover, many of the therapeutic effects of activating the Mre11 pathway are UV-mimetic, and UV is known to activate both ATR and DNA-PK but not ATM.

1. A method of screening for a modulator of Mre11 comprising:
(a) contacting candidate modulators with Mre11 in vitro in the presence of a nucleic acid substrate for Mre11; and
(b) measuring the hydrolysis of said substrate, whereby a modulator is identified by altering hydrolysis of said substrate compared to a control.

2. The method of claim 1 wherein said nucleic acid substrate is an oligonucleotide with at least 50% nucleotide sequence identity with (TTAGGG)ₙ, wherein n=1 to 20.

3. The method of claim 1 wherein hydrolysis of said nucleic acid substrate is measured by UV absorbance or release of a radiolabel.

4. A method of screening for an agent that specifically binds to Mre11 comprising:
(a) contacting candidate agents with Mre11; and
(b) determining whether a candidate agent specifically binds to Mre11.

5. The method of claim 4 wherein Mre11 is attached to a solid support.

6. A method of screening for a modulator of Mre11 comprising:
(a) providing a cell that expresses Mre11;
(b) contacting candidate modulators with said cell under conditions in which the modulator is taken up by the cell; and
(c) measuring a property of said cells selected from the group consisting of cellular proliferation, cellular viability, cellular morphology, SA-β-Gal activity and phosphorylation of p53 or p95, whereby a modulator is identified by altering said property compared to a control.

7. The method of claim 6 wherein said candidate modulators specifically bind to Mre11.

8. The method of claim 6 wherein said Mre11 is a fragment, homolog, analog or variant of Mre11.

9. The method of claim 8 wherein said fragment, homolog, analog or variant of Mre11 has exonuclease activity.

10. The method of claim 6 wherein the property of said cell is cellular proliferation.

11. The method of claim 6 wherein the property of said cell is cellular viability.

12. The method of claim 6 wherein the property of said cell is cellular morphology.

13. The method of claim 6 wherein the property of said cell is SA-β-Gal activity.

14. The method of claim 6 wherein the property of said cell is phosphorylation of p53 or p95.

15. The method of claim 6 wherein said cell is a cancer cell.

16. The method of claim 15 wherein the telomeres of said cell are maintained by telomerase reverse transcriptase or the ALT pathway.

17. The method of claim 8 wherein said cell is a cancer cell.

18. The method of claim 17 wherein the telomeres of said cell are maintained by telomerase reverse transcriptase or the ALT pathway.

19. The method of claim 15 wherein said candidate modulators are selected from the group consisting of carbohydrates, monosaccharides, oligosaccharides, polysaccharides, amino acids, peptides, oligopeptides, polypeptides, proteins, nucleosides, nucleotides, oligonucleotides, polynucleotides, lipids, retinoids, steroids, glycopeptides, glycoproteins, proteoglycans, and small organic molecules.

20. The method of claim 19 wherein the telomeres of said cell is maintained by telomerase reverse transcriptase or the ALT pathway.

21. The method of claim 17 wherein said candidate modulators are selected from the group consisting of carbohydrates, monosaccharides, oligosaccharides, polysaccharides, amino acids, peptides, oligopeptides, polypeptides, proteins, nucleosides, nucleotides, oligonucleotides, polynucleotides, lipids, retinoids, steroids, glycopeptides, glycoproteins, proteoglycans, and small organic molecules.

22. The method of claim 21 wherein the telomeres of said cell is maintained by telomerase reverse transcriptase or the ALT pathway.

23. A method of screening for modulator of tankyrase comprising:
(a) contacting candidate modulators with tankyrase in vitro in the presence of a substrate for tankyrase; and
(b) measuring the ribosylation of said substrate, whereby a modulator is identified by altering ribosylation of said substrate compared to a control.

24. The method of claim 23 wherein said substrate is a peptide or polypeptide.

25. The method of claim 24 wherein said substrate is TRF1.

26. The method of claim 23 wherein ribosylation of said substrate is measured by UV absorbance or labeling of said substrate.

27. A method of screening for an agent that specifically binds to tankyrase comprising:
(a) contacting candidate binders with tankyrase; and
(b) determining whether a candidate agent specifically binds to tankyrase.

28. The method of claim 27 wherein tankyrase is attached to a solid support.

29. A method of screening for modulator of tankyrase comprising:
(a) providing a cell that expresses tankyrase;
(b) contacting candidate modulators with said cell under conditions in which the modulator is taken up by the cell; and
(c) measuring a property of said cells selected from the group consisting of cellular proliferation, cellular viability, cellular morphology, SA-β-Gal activity and phosphorylation of p53 or p95, whereby a modulator is identified by altering said property compared to a control.

30. The methods of claim 29 wherein said candidate modulators specifically bind to tankyrase.

31. The method of claim 23 wherein said tankyrase is a fragment, homolog, analog or variant of tankyrase that has ribosylation activity.

32. The method of claim 31 wherein said fragment, homolog, analog or variant of tankyrase has ribosylase activity.

33. The method of claim 29 wherein the property of said cell is cellular proliferation.

34. The method of claim 29 wherein the property of said cell is cellular viability.

35. The method of claim 29 wherein the property of said cell is cellular morphology.

36. The method of claim 29 wherein the property of said cell is SA-β-Gal activity.

37. The method of claim 29 wherein the property of said cell is phosphorylation of p53 or p95.

38. The method of any of claim 37 wherein said cell is a cancer cell.

39. The method of claim 38 wherein the telomeres of said cell is maintained by telomerase reverse transcriptase or the ALT pathway.

40. The method of claim 31 wherein said cell is a cancer cell.

41. The method of claim 40 wherein the telomeres of said cell is maintained by telomerase reverse transcriptase or the ALT pathway.

42. The method of claim 38 wherein said candidate modulators are selected from the group consisting of carbohydrates, monosaccharides, oligosaccharides, polysaccharides, amino acids, peptides, oligopeptides, polypeptides, proteins, nucleosides, nucleotides, oligonucleotides, polynucleotides, lipids, retinoids, steroids, glycopeptides, glycoproteins, proteoglycans, and small organic molecules.

43. The method of claim 42 wherein the telomeres of said cell is maintained by telomerase reverse transcriptase or the ALT pathway.

44. The method of claim 40 wherein said candidate modulators are selected from the group consisting of carbohydrates, monosaccharides, oligosaccharides, polysaccharides, amino acids, peptides, oligopeptides, polypeptides, proteins, nucleosides, nucleotides, oligonucleotides, polynucleotides, lipids, retinoids, steroids, glycopeptides, glycoproteins, proteoglycans, and small organic molecules.

45. The method of claim 44 wherein the telomeres of said cell is maintained by telomerase reverse transcriptase or the ALT pathway.

46. A method of screening for a modulator of MRN complex formation comprising:
(a) contacting candidate modulators with Mre11, Rad50 and Nbs1 in vitro; and
(b) measuring the formation of the MRN complex, whereby a modulator is identified by altering formation of the MRN complex compared to a control.

47. The method of claim 46 wherein candidate modulators are contacted with Mre11, Rad50 and Nbs I in the presence of a nucleic acid substrate or inhibitor of Mre11.

48. The method of claim 47 wherein said nucleic acid is an oligonucleotide with at least 50% nucleotide sequence identity with (TTAGGG)ₙ, wherein n=1 to 20.

49. The method of claim 46 wherein formation of the MRN complex is measured by centrifugation, coprecipitation or nondenaturing electrophoresis.

50. A method of screening for a modulator of the DNA damage pathway comprising:
(a) providing a cell that expresses Mre11 and tankyrase;
(b) contacting candidate modulators with said cell in the presence of an oligonucleotide under conditions in which the modulator is taken up by the cell; and
(c) measuring a property of said cells selected from the group consisting of cellular proliferation, cellular viability, cellular morphology, SA-β-Gal activity and phosphorylation of p53 or p95, whereby a modulator is identified by altering said property compared to a control,
wherein said oligonucleotide has at least 50% nucleotide sequence identity with (TTAGGG)ₙ, wherein n=1 to 20.

51. The method of claim 50 wherein said Mre11 is a fragment, homolog, analog or variant of Mre11.

52. The method of claim 51 wherein said fragment, homolog, analog or variant of Mre11 has exonuclease activity.

53. The method of claim 50 wherein said tankyrase is a fragment, homolog, analog or variant of tankyrase.

54. The method of claim 53 wherein said fragment, homolog, analog or variant of tankyrase has ribosylation activity.

55. The method of claim 50 wherein the property of said cell is cellular proliferation.

56. The method of claim 50 wherein the property of said cell is cellular viability.

57. The method of claim 50 wherein the property of said cell is cellular morphology.

58. The method of claim 50 wherein the property of said cell is SA-β-Gal activity.

59. The method of claim 50 wherein the property of said cell is phosphorylation of p53 or p95.

60. The method of claim 59 wherein said cell is a cancer cell.

61. The method of claim 61 wherein the telomeres of said cell is maintained by telomerase reverse transcriptase or the ALT pathway.

62. The method of claim 50 wherein said candidate modulators are selected from the group consisting of carbohydrates, monosaccharides, oligosaccharides, polysaccharides, amino acids, peptides, oligopeptides, polypeptides, proteins, nucleosides, nucleotides, oligonucleotides, polynucleotides, lipids, retinoids, steroids, glycopeptides, glycoproteins, proteoglycans, and small organic molecules.

63. A method of treating cancer comprising administering to a subject in need of such treatment a composition comprising an activator of Mre11, tankyrase, the DNA damage pathway or MRN complex formation.

64. A method of inducing apoptosis comprising administering to a subject in need of such treatment a composition comprising an activator of Mre11, tankyrase, the DNA damage pathway or MRN complex formation.

65. A method of inducing cellular senescence comprising administering to a subject in need of such treatment a composition comprising an activator of Mre11, tankyrase, the DNA damage pathway or MRN complex formation.

66. A method of inhibiting tanning comprising administering to a subject in need of such treatment a composition comprising an activator of Mre11, tankyrase, the DNA damage pathway or MRN complex formation.

67. A method of promoting cellular differentiation comprising administering to a subject in need of such treatment a composition comprising an activator of Mre11, tankyrase, the DNA damage pathway or MRN complex formation.

68. A method of promoting immunosuppression comprising administering to a subject in need of such treatment a composition comprising an activator of Mre I1, tankyrase, the DNA damage pathway or MRN complex formation.

69. The method of claim 68 wherein the activator is an oligonucleotide activator of Mre11 with at least 50% nucleotide sequence identity with (TTAGGG)ₙ and at least the first x 3′-nucleotide linkages are hydrolyzable by a 3′ to 5′ nuclease, wherein n=1 to 20, and wherein x is from about 1 to about 10.

70. A method of inhibiting apoptosis comprising administering to a subject in need of such treatment a composition comprising an inhibitor of Mre11, tankyrase, the DNA damage pathway or MRN complex formation.

71. A method of inhibiting cellular senescence comprising administering to a subject in need of such treatment a composition comprising an inhibitor of Mre11, tankyrase, the DNA damage pathway or MRN complex formation.

72. A method of promoting growth comprising administering to a subject in need of such treatment a composition comprising an inhibitor of Mre11, tankyrase, the DNA damage pathway or MRN complex formation.

73. A method of promoting tanning comprising administering to a subject in need of such treatment a composition comprising an inhibitor of Mre11, tankyrase, the DNA damage pathway or MRN complex formation.

74. A method of inhibiting cellular differentiation comprising administering to a subject in need of such treatment a composition comprising an inhibitor of Mre11, tankyrase, the DNA damage pathway or MRN complex formation.

75. A method of reducing cancer treatment side effects comprising administering to a subject in need of such treatment a composition comprising an inhibitor of Mre11, tankyrase, the DNA damage pathway or MRN complex formation.

76. The method of claim 75 wherein the composition is given in combination with chemotherapy or ionizing radiation.

77. The method of claim 76 wherein the inhibitor is an oligonucleotide inhibitor of Mre11 with at least 50% nucleotide sequence identity with (TTAGGG)ₙ and at least the first x 3′-nucleotide linkages are hydrolyzable by a 3′ to 5′ nuclease, wherein n=1 to 20, and wherein x is from about 0 to about 10.

78. A composition comprising an oligonucleotide with at least 50% nucleotide sequence identity with (TTAGGG)ₙ and at least one nonhydrolyzable internucleotide linkage,wherein at least the first x 3′-nucleotide linkages are hydrolyzable by a 3′ to 5′ nuclease, wherein n=1 to 20, and wherein x is from about 0 to about 10.

79. The composition of claim 78 wherein the 3′ to 5′ nuclease is Mre11.

80. The composition of claim 78 wherein the oligonucleotide has at least 50% nucleotide sequence identity with TTAGGG.

81. The composition of claim 80 wherein the oligonucleotide or thereof has the sequence GTTAGGGTTAG.

82. The composition of claim 78 wherein the nonhydrolyzable linkage is a phosphorothioate.

83. The composition of claim 78 wherein the oligonucleotide is a PNA.
